(19) United States
(12) Patent Application Publication
(10) Pub. No.: US 2024/0158439 A1
(43) Pub. Date: May 16, 2024

(54) INHIBITORS OF ANTIGEN PRESENTATION BY MHC CLASS II HLA-DRB1*15:01 MOLECULES WITH ENHANCED METABOLIC STABILITY

(71) Applicant: Provid Pharmaceuticals Inc., Monmouth Junction, NJ (US)

(72) Inventors: Gary L. OLSON, Mountainside, NJ (US); Christopher R. SELF, West Caldwell, NJ (US); Yanjun SUN, Kendall Park, NJ (US)

(21) Appl. No.: 18/280,501

(22) PCT Filed: Mar. 9, 2022
(86) PCT No.: PCT/US2022/019445

§ 371 Date: Sep. 6, 2023

Related U.S. Application Data
(60) Provisional application No. 63/163,172, filed on Mar. 19, 2021.

(51) Int. Cl. C07K 7/06

(52) U.S. Cl. CPC C07K 7/06

(57) ABSTRACT

Provided are compounds, pharmaceutical compositions containing such compounds and methods for using the compounds the treatment or prevention of autoimmune diseases and disorders associated with antigen presentation by MHC Class II HLA-DRB1*15:O1 and furthermore, have improved metabolic stability compared to previous inhibitors.

### FIELD OF THE INVENTION

The invention relates to novel compounds, pharmaceutical compositions containing such compounds and methods for their use. In particular, the compounds of the invention are useful for the treatment or prevention of autoimmune diseases and disorders associated with antigen presentation by MHC Class II HLA-DRB1*15:01 and have enhanced metabolic stability.

All references and patents cited to or relied upon below are expressly incorporated herein by reference in their entirety.

### BACKGROUND OF THE INVENTION

As previously described (Olson, et al, U.S. Pat. 7,439,231, expressly incorporated herein by reference) host immune responses are commonly classified into two distinguishable groups, cellular and humoral. Cellular immunity is mediated by T lymphocytes or T cells and protects against virally infected cells, fungi, parasites, and foreign tissue. Humoral immunity, which is mediated by B lymphocytes or B cells through the production of antibodies, is most effective against bacterial infections and the extracellular phases of viral infections. D. Voet & J. Voet, Biochemistry 1208 (2d ed., Wiley 1995).

Cellular immune response cascades lead to the destruction of pathogens through: 1) the uptake of antigens derived from pathogens by cellular antigen presenting cells or macrophages, 2) the processing or fragmentation of the antigen within the antigen presenting cell, 3) the binding to and presentation of the fragmented antigen to cell-surface proteins known as the major histocompatibility complex (“MHC”) proteins and 4) binding of the MHC protein/antigen complex by T cells that are induced to propagate, thereby eliciting an effective immune response against the specific antigen and leading to destruction of the pathogen. In autoimmune disorders, this cellular immune response cascade recognizes self-derived species as autoantigens, effectively leading to the destruction of host peptides, cells, and tissues resulting in autoimmune disease pathology. The process by which the cellular immune system recognizes and initiates a response to antigens, both foreign and self, via the MHC system has been a focus of much research in recent years.

MHC proteins have been classified into two groups, referred to as Class I and Class II MHC proteins, that are structurally and functionally similar. D. Voet & J. Voet, Id. Macrophages exhibiting Class I MHC proteins or Class II MHC proteins that are complexed with an antigen on their surface are bound by cytotoxic T cells or helper T cells, respectively. As a result of this binding event, T cells are induced to proliferate and trigger an immune response against the antigen. The role of MHC proteins is to present the antigen on the surface of the cell so that they can be recognized by T cells. In humans, the Class I MHC proteins are encoded by three separate genetic loci, HLA-A, HLA-B, and HLA-C. There are also three heterodimeric human Class II MHC proteins whose alpha and beta chains are encoded by genes designated HLA-DP, HLA-DQ, and HLA-DR. Both Class I and Class II MHC genes are highly polymorphic, giving rise to the variance between individuals in the population. Id. Because of the key role of the antigen/MHC complex in the activation of T cells, inhibition of antigen binding to MHC molecules has been a goal of research in autoimmune diseases. Id.

In autoimmune diseases, inappropriate triggering of T cell responses by MHC molecule— “self antigen” complexes leads to destruction of normal tissues. Individuals inherit MHC genes of the HLA-DR, -DP, and -DQ haplotypes, and these have been linked to specific autoimmune diseases and autoantigens such as multiple sclerosis and rheumatoid arthritis. In each of these cases, patients diagnosed with the autoimmune disease carry an associated MHC gene.

In multiple sclerosis, the strongest and most significant genetic association is with the HLA-DRB1*15:01 gene (also referred to by earlier nomenclature as DR2b and DR15) (Link, et al. (2012) PLoS ONE 7: e36779). In Goodpasture's disease (anti-GBM disease) over 90% have the DRB1*15:01 gene (Ooi, et al., (2013) J Am Soc Nephrol 24: 419-431). In rheumatoid arthritis, over 90% of patients have either DRB*0101 (DR1), 0401 (DR4), 0404, or 0405 genes (Falcioni, et al., (1999) Nature Biotechnol. 17: 562-567).

Design of inhibitors of the cellular immune response has been a fundamental goal of research that aims to prevent T cell proliferation in autoimmune disease. Yusuf-Makagiansar et. al. (2002)Med. Res. Rev. 22(2):146-167, Adorini, et al., (1988) Nature, 334, 623-625. Inhibition based on blocking HLA DRB1*01:01 and DRB1*04:01 alleles associated, for example, with rheumatoid arthritis by various 7-mer peptides has been described (Falcioni, et al., (1999) Nature Biotech, 17: 562-567 and refs therein). In that work, peptide-based inhibitors that bound to DR1 or DR4 were identified. These demonstrated cellular activity when the peptide-based inhibitors were stabilized by changes that prevented degradation of the peptide backbone by proteolyic enzymes of the cathepsin class that reside in the endosome to process protein antigens.

The molecular interactions between antigenic peptides or peptide-based inhibitors and MHC molecules have been defined in a series of high-resolution crystal structures. DRB1*0101 with HA antigen: Stern et al. (1994) Nature 368:215-221; DRB1*0401 with collagen II antigen: Dessen A., et al. (1997) Immunity. 7:473-81; DRB1*0401 with inhibitors: Bolin, et al. (2000) J Med. Chem. 43, 2135-48; DRB1*15:01 with myelin peptide antigen: Smith et al. (1998) J Exp. Med. 188:1511-1520. The general observations for these complexes include the extended, poly(proline) II helical conformation of the peptide backbone, the presence of pockets (that bind so-called anchor residues) along the chain, and networks of hydrogen bonds between the peptide backbone and side chains of the MHC molecules that line the binding site.

In other work, requirements for peptide binding have been assessed using structure-activity studies (Hammer, J. et al. (1993) Cell 74:197-203) with peptide phage display libraries.

Multiple sclerosis is a chronic demyelinating autoimmune disease of the central nervous system that afflicts over 2 million patients worldwide, with approximately 1 million patients in the U.S. With the exception of trauma, multiple sclerosis is the leading cause of neurologic disability in early to middle adulthood. The disease is typically chronic. The most common form of multiple sclerosis is relapsing-remitting MS (RRMS; about 85% of patients). Other forms include juvenile and prodromal MS, clinically and radiologically isolated syndromes (CIS/RIS), secondary progressive MS (SPMS; about 15% of RRMS patients go on to develop SPMS), and primary progressive MS (PPMS; about 5% have continuous disease progression without remissions). All of these forms share the disease association genetics with the MHC class II DRB1*15:01 although progressive forms appear to have extended pathology resulting from additional immune and inflammatory mechanisms. MS is incapacitating and affects multiple body systems. While there are multiple therapeutic drugs acting on various disease pathways in use to treat MS, the balance between efficacy and safety is unsatisfactory and none of the current approaches is tailored to the genetics of the MS patient. Accordingly, there remains a need for an effective treatment for multiple sclerosis in addition to other autoimmune diseases.

Multiple sclerosis is genetically associated with the major histocompatibility complex class II (MHC class II) gene and more specifically with the MHC class II HLA-DRB1*15:01 molecule (DR2b). The DRB1*15:01 molecule binds and presents antigenic peptide fragments of myelin nerve sheath proteins that insulate nerves to autoreactive T cells. These T cells are triggered to proliferate when they encounter a myelin antigen presented in complex with DRB1*15:01 and mount an attack on the myelin insulation, resulting in the disease pathology. In people of northern European descent, approximately 70% of multiple sclerosis patients carry the HLA-DRB1*1501 gene. In the US overall, the percentage is from 40-60%. (Giordano, M. et al. (2002) Am. J. Pharmacogenomics 2:37-58). Accordingly, compounds with the ability to inhibit antigen binding by the MHC class II HLA-DRB1*15:01 are expected to be effective therapeutics for treating or preventing multiple sclerosis. Other autoimmune diseases are associated with antigen binding to DRB1*15:01; for example, anti-GBM disease (Goodpasture's disease).

In the case of inhibitors of DRB1*15:01 of value in the treatment of multiple sclerosis and related autoimmune diseases, the crystal structure of DRB1*15:01 in complex with a myelin peptide antigen published by Smith and Wiley demonstrated features in common and unique differences between the DRB1*04:01 and DRB1*01:01 MHC class II molecules (Smith et al. (1998) J. Exp. Med. 188:1511-1520).

The structural requirements for binding of a compound to DRB1*15:01 molecules were also elucidated by probing with peptides related to myelin basic protein (“MBP”). Wucherpfennig, et al. (1994) J. Exp. Med. 179:279-290. The immunodominant MBP (84-102) peptide was found to bind with high affinity to DRB1*15:01 and DRB5*01:01 molecules of the disease associated DR2 haplotype. Other peptide segments that overlapped with this peptide were also critical for binding to these molecules. It was demonstrated that hydrophobic residues (Va189 and Phe92) in the MBP (88-95) segment were critical for peptide binding to DRB1*15:01 molecules and that hydrophobic and charged residues (Phe92, Lys93) in the MBP (89-101/102) sequence contributed to DRB5*01:01 binding. The DRB1*15:01 structure also showed unique features of the binding site due to a larger P4 binding pocket in DRB1*15:01 compared to other DR molecules that could be exploited for the design of inhibitors with high selectivity for DRB1*15:01.

Goodpasture's disease (anti-glomerular basement membrane (GBM) disease) is a rapidly progressive glomerulonephritis autoimmune disease strongly associated with HLA-DRB1*15:01. The immunodominant T-cell epitope α3₁₃₅₋₁₄₅ is restricted to HLA-DRB1*15:01. A DRB1*15:01-specific inhibitor has potential as a targeted therapy that may be superior to general immunosuppresants. Hunyn, et al., (Journal of Autoimmunity 103 (2019) 102276 demonstrated that specific inhibition of HLA-DRB1*1501 by PV-267 (Ac-V-Chg-R-Tic-F-NH₂) attenuates autoreactivity to the Goodpasture antigen (non-collagenous domain of the α3 chain of type IV collagen, α3(IV)NC1). HLA-DRB1*15:01 inhibition also attenuates autoimmunity and glomerular injury in experimental autoimmune anti-GBM disease in DRB1*15:01 transgenic mice and demonstrates the potential for specific MHCII inhibitors as a targeted therapy for anti-GBM (Goodpasture's) disease.

The demonstrations of efficacy in disease models of multiple sclerosis and Goodpasture's disease establishes that the blockade of the MHC class II DRB1*15:01 binding site by inhibitors is useful to prevent or treat disease pathology independent of the triggering antigen (e.g., myelin fragments for MS/EAE or the type IV collagen α3₁₃₅₋₁₄₅ antigen in Goodpasture's disease.

Previous work described inhibitors of MHC class II DRB1*15:01 that block antigen binding and are active in human cellular immunology assays and are effective in animal models of MS in DRB1*15:01 transgenic mice. (Olson, et al., U.S. Pat. Nos. 7,439,231, 8,222,215, 8,598,312, expressly incorporated herein by reference).

These compounds that block antigen binding to DRB1*15:01 and inhibit presentation to T cells, the first and key steps in the immune cascade, are the most appropriate for the treatment of RRMS, early MS, and for the prevention of progression from RRMS to SPMS. They may also be of value in combination with other treatments. Treatment with an inhibitor of antigen binding to DRB1*15:01 would be relevant to all patients who have this disease-associated gene. Unlike other disease-modifying MS drugs, the specificity of inhibitors for DRB1*15:01 promises a superior safety profile in that the inhibitors block only the disease associated MHC allele and do not otherwise compromise the normal immune system. The gene and MS are most common in individuals of Northern European descent. In Asia and Africa, MS is present but less frequent due to the low percentage of patients with the DRB1*15:01 gene (Walton, C., et al. “Rising prevalence of multiple sclerosis worldwide: Insights from the Atlas of MS, third edition.” Multiple sclerosis (Houndmills, Basingstoke, England) vol. 26,14 (2020): 1816-1821).

A series of inhibitors of antigen binding to MHC class II DR2b (DRB1*15:01) were described in the U.S. Pat. Nos. 7,439,231, 8,222,215, and 8,598,312, expressly incorporated herein by reference, with claims allowed for both composition of matter and methods of use. Their activity and utility were demonstrated in multiple assays suggesting value in the treatment of autoimmune diseases including multiple sclerosis (MS). The research background and scientific results are summarized in the publication: Ji N, Somanaboeina A, Dixit A, Kawamura K, Hayward N J, Self C, Olson G L, Forsthuber T G. Small molecule inhibitor of antigen binding and presentation by HLA-DR2b as a therapeutic strategy for the treatment of multiple sclerosis. J Immunol. 2013 Nov 15;191(10):5074-84. This work described inhibitors of MHC class II DRB1*15:01 that block antigen binding and are active in human cellular immunology assays and are effective in animal models of MS in DRB1*15:01 transgenic mice.

### SUMMARY OF THE INVENTION

Provided as an embodiment of the invention is a compound of the formula (I):

K₁—P₁—P₂—P₃—P₄—P₅—K₂ (I),

or a pharmaceutically acceptable salt thereof,
wherein:
K₁ is an N-capping group;
P₁-P₅ are substituents that, when present, are coupled by amide bonds and are each independently amino acyl groups of a natural amino acid or a non-natural amino acid; and
K₂ is a substituent in which the C-terminus is an oligoalkoxyalkyl amide group.

Also provided as embodiments of the invention are pharmaceutical compositions containing such compounds and methods for using the compounds the treatment or prevention of autoimmune diseases and disorders associated with antigen presentation by MEW Class II HLA-DRB1*15:01.

### BRIEF DESCRIPTION OF THE DRAWINGS

FIG. 1 shows activity data of the amide PV-267 and the acid PV-623 inhibiting IFN-gamma response in the human cellular immunology assay (10,000 BC3 T cells+0.5×10⁶ autologous hPBMCs from DRB1*15:01-positive MS patient (mean±SEM).

FIG. 2 shows cellular immunology results for two novel substituted amide analogs compared to PV-267.

### DETAILED DESCRIPTION OF THE INVENTION

In the US patents cited (see Table 1), compounds were defined by the generic formula

K₁—P₁—P₂—P₃—P₄—P₅—P₆—P₇—P₈—P₉—P₁₀—K₂ (A)

with definitions as described in the patents shown in Table 1:

TABLE 1

| | | | |
|---|---|---|---|
| Title | Inhibition of Antigen | Methods of Use of | Inhibitors of Antigen |
| | Presentation by MHC | Inhibitors of Inhibition | Presentation by MHC |
| | Class II Molecules and | of Antigen Presentation | Class II Molecules |
| | Methods of Use Thereof | by MHC Class II Molecules | |
| U.S. Pat. No. | 7,439,231, expressly | 8,222,215, expressly | 8,598,312, expressly |
| | incorporated herein | incorporated herein | incorporated herein |
| | by reference | by reference | by reference |
| Issue Date | Oct. 21, 2008 | Jul. 17, 2012 | Dec. 3, 2013 |
| Date Filed | Feb. 13, 2004 | Sep. 5, 2008 | Jun. 15, 2012 |
| Initial Filing Date | Feb. 13, 2004 | Feb. 13, 2004 | Feb. 13, 2004 |

Among these compounds, sequences of 5 residues, i.e., compounds of the formula

K₁—P₁—P₂—P₃—P₄—P₅—K₂ (B)

were found to be to be as potent in competitive binding assays as longer sequences. The preferred sequences were described as peptide amides (K₂=NH₂) but the peptide acid (K₂=OH) was also active. Table 2 lists exemplary compounds with 5 residues that show activity in binding assays with IC50 values under 250 nM (entries 1-45) and 5 reference compounds (entries 46-50).

TABLE 2
5-Residue DRB1*15:01 inhibitors of the Formula (B)

| Entry | PVD | K1 | P1 | P2 | P3 | P4 | P5 | K2 | Avg IC50 |
|---|---|---|---|---|---|---|---|---|---|
| 1 | 633 | NH2-Gly | V | Chg | R | Tic | F | NH2 | 1.0 |
| 2 | 579 | Ac | V | Chg | R | Tic | Trp | NH2 | 1.3 |
| 3 | 590 | Ac | t-Bug | Chg | R | Tic | F | NH2 | 1.3 |
| 4 | 493 | Ac | V | Chg | R | Tic | (4Indole)A | NH2 | 1.5 |
| 5 | 635 | NH2-Abu | V | Chg | R | Tic | F | NH2 | 2.0 |
| 6 | 638 | NH2-Beta | V | Chg | R | Tic | F | NH2 | 2.0 |
| | | Ala | | | | | | | |
| 7 | 664 | NH2-Beta | V | Chg | Cit | Tic | F | NH2 | 2.0 |
| | | Ala | | | | | | | |
| 8 | 478 | Ac | V | Chg | R | Tic | (3-BnThienyl)A | NH2 | 2.1 |
| 9 | 574 | Ac | V | Chg | R | Tic | 4-F Phe | NH2 | 2.3 |
| 10 | 663 | NH2-Abu | V | Chg | Cit | Tic | F | NH2 | 2.5 |
| 11 | 267 | Ac | V | Chg | R | Tic | F | NH2 | 2.5 |
| 12 | 636 | NH2-Sar | V | Chg | R | Tic | F | NH2 | 3.0 |
| 13 | 676 | Ac | V | Chg | R | Tic | 4-Indazole | NH2 | 3.0 |
| 14 | 642 | Ac | V | Chg | Nor Cit | Tic | F | NH2 | 4.0 |
| 15 | 662 | NH2-Sar | V | Chg | Cit | Tic | F | NH2 | 4.0 |
| 16 | 591 | Ac | nor V | Chg | R | Tic | F | NH2 | 4.1 |
| 17 | 592 | Ac | T(OMe) | Chg | R | Tic | F | NH2 | 4.1 |
| 18 | 593 | Ac | V | Chg | R | Tic | 3,4 diCl Phe | NH2 | 4.1 |
| 19 | 661 | NH2-Gly | V | Chg | Cit | Tic | F | NH2 | 5.0 |
| 20 | 562 | Ac | V | Chg | nor Arg | Tic | F | NH2 | 5.3 |
| 21 | 810 | Ac | V | Chg | GuanPipG | Tic | F | NH2 | 6.0 |
| 22 | 573 | Ac | V | Cpg | R | Tic | F | NH2 | 6.7 |
| 23 | 640 | Ac | V | Chg | Homo Cit | Tic | F | NH2 | 7.0 |
| 24 | 594 | Ac | V | Indgly | R | Tic | F | NH2 | 14.0 |
| 25 | 625 | Ac | V | Chg | R | Tic | F | NHCH3 | 16.0 |
| 26 | 629 | Ac | V | Indgly | R | Tic | F | NHCH3 | 17.0 |
| 27 | 833 | NH2-Beta | V | Chg | (4-Thiaz) | Tic | F | NH2 | 17.0 |
| | | Ala | | | A | | | | |
| 28 | 626 | Ac | V | Indgly | R | Tic | F | morpholine | 19.0 |
| 29 | 839 | Ac | V | Chg | 2 Amino | Tic | F | NH2 | 25.0 |
| | | | | | His | | | | |
| 30 | 623 | Ac | V | Chg | R | Tic | F | OH | 26.0 |
| 31 | 580 | Ac | V | Chg | H | Tic | F | NH2 | 28.0 |
| 32 | 588 | Ac | V | Chg | R | homo | F | NH2 | 28.0 |
| | | | | | | Pro | | | |
| 33 | 526 | Ac | Thr | Chg | R | Tic | F | NH2 | 29.0 |
| 34 | 476 | | Ac | Chg | R | Tic | (4Indole)A | NH2 | 30.1 |
| 35 | 630 | Ac | V | Chg | R | Tic | F | morpholine | 33.0 |
| 36 | 634 | NH2-BTA | V | Chg | R | Tic | F | NH2 | 37.0 |
| 37 | 812 | NH2-Beta | V | Chg | Pyrimi- | Tic | F | NH2 | 39.0 |
| | | Ala | | | dinyl | | | | |
| 38 | 560 | Ac | V | Chg | A | Tic | F | NH2 | 43.0 |
| 39 | 507 | | Ac | Chg | R | Tic | Trp | NH2 | 48.0 |
| 40 | 477 | | Ac | Chg | R | Tic | (3-BnThienyl)A | NH2 | 82.0 |
| 41 | 618 | Ac | V | Chg | R | Tic | 4-Benzimidazole | NH2 | 96.0 |
| 42 | 628 | Ac | V | Indgly | R | Tic | F | morpholine | 104.0 |
| 43 | 782 | NH2-Beta | V | Chg | F | F | F | NH2 | 150.0 |
| | | Ala | | | | | | | |
| 44 | 624 | Ac | V | Indgly | R | Tic | F | OH | 161.0 |
| 45 | 508 | | Ac | Chg | R | Tic | 1 Me-Trp | NH2 | 213.0 |
| 46 | 708 | Ac | V | Chg | R | Tic | Phenethylamide | | 464.0 |
| 47 | 589 | Ac | V | Chg | R | Oic | F | NH2 | 474.0 |
| 48 | 73* | Ac | V | V | R | F | F | NH2 | 2370.0 |
| 49 | 617* | Ac | V | Chg | R | Tic | 2-Quinolyl Ala | NH2 | 3450.0 |
| 50 | 119* | | Ac | V | R | F | F | NH2 | 5000.0 |

*Reference

Among the sequences in Table 2, PV-267 (Ac—Val—Chg—Arg—Tic—Phe—NH₂) has been studied in multiple biological assays and has demonstrated inhibition of antigen binding, T cell activation in human cellular, and efficacy in animal models of multiple sclerosis (EAE in DRB 1*15:01 transgenic mice). In this work, PV-267 is demonstrated to be a promising candidate for the treatment of MS. Additional compounds were shown to be active in binding assays, pharmacokinetics tests, enzyme stability, in human cellular immunology assays using MS patient cells, and in the EAE model of MS in DRB1*15:01 transgenic mice (Ji, N, et al., op. cit.). Similar activity was observed in models of anti-GBM (Goodpasture's) disease.

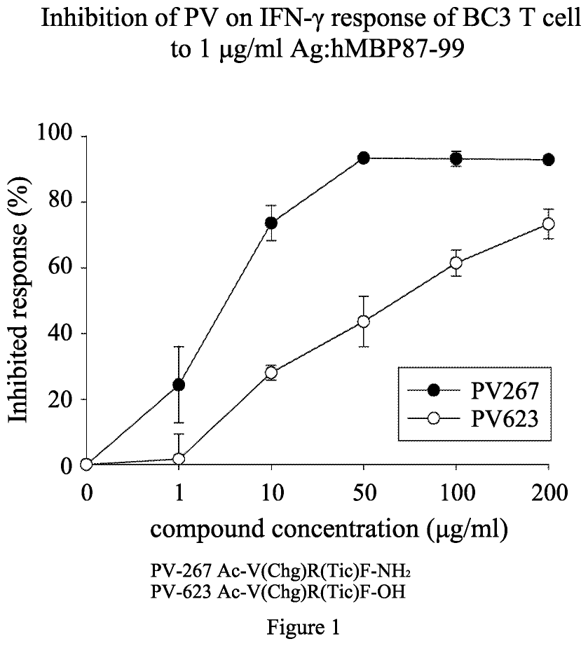

In studies of the pharmacokinetics of PV-267 in rats and mice, it was found that in vivo, PV-267 (which has a C-terminal amide group) is significantly converted to the C-terminal acid PV-623 (Table 3) by metabolic transformation. The acid metabolite PV-623 retains activity in binding to DRB1*15:01 and blocking antigen-induced T cell activation and production of interferon gamma in cells from a DRB1*15:01 positive human MS patient as well as in inhibition of antigen binding, but the acid PV-623 is 5-fold to 13-fold less potent than the amide PV-267 (FIG. 1).

TABLE 3
PV-267 (C-terminal amide) and PV-623 (C-terminal acid)

| | |
|---|---|
| PV-267 | 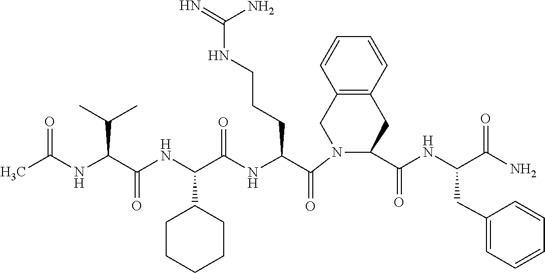 [SMILES: CC(=O)N[C@H](C(=O)N[C@H](C(=O)N[C@@H](CCCNC(=N)N)C(=O)N1Cc2ccccc2C[C@H]1C(=O)N[C@@H](Cc1ccccc1)C(N)=O)C1CCCCC1)C(C)C] |
| | Ac-V-(Chg)-R-(Tic)-F—NH₂ |
| PV-623 (metabolite of PV-267) | 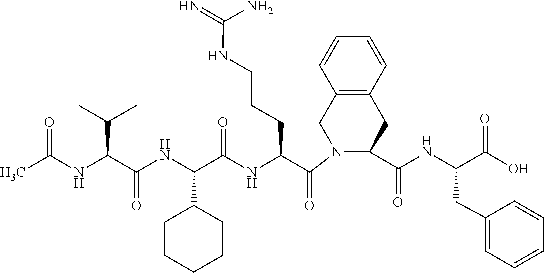 [SMILES: CC(=O)N[C@H](C(=O)N[C@H](C(=O)N[C@@H](CCCNC(=N)N)C(=O)N1Cc2ccccc2C[C@H]1C(=O)N[C@@H](Cc1ccccc1)C(=O)O)C1CCCCC1)C(C)C] |
| | Ac-V-(Chg)-R-(Tic)-F—OH |

The observation that the C-terminal acid PV-623 was less potent than the amide PV-267 indicated that a novel variant of the structure that was not metabolized to a less active form would be highly desirable. However, previous studies in the series had shown that replacement of the primary amide (as in PV-267) by an N-methyl amide (PV-625) led to an 8-fold loss in binding affinity, leading to the conclusion that simple substitution of the amide was not of interest for further study.

Unexpectedly, substitution of the amide with an oligoalkoxyalkyl C-cap group, such as a methoxyethylamide group (K₂=NHCH₂CH₂OCH₃), produced an analog PV-3212 (see structure in Table 4) that was remarkably (a) more potent than the simple amide (PV-267) in human cellular assays and (b) was not metabolized to the acid PV-623 in mouse PK studies. Shown in Table 4 is the mouse PK data for PV-3212 compared to PV-267.

TABLE 4
Mouse PK data for PV-267 and stabilized analog PV-3212.

| | Mean Plasma Concentration. | | | | |
|---|---|---|---|---|---|
| Compound | 0.25 hr | | 0.5 hr | | t_{1/2} |
| PV-267 (Amide) | 275 | μg/mL | 91.5 | μg/mL | |
| Ac-V(Chg)R(Tic)F—NH₂ | | | | | |
| PV-623 (Acid metabolite) | 169 | μg/mL | 65.3 | μg/mL | |
| Ac-V(Chg)R(Tic)F—OH | | | | | |
| PV-3212 (2^{nd} Gen) | 741 | μg/mL | 195 | μg/mL | 3.23 hr |
| Ac-V(Chg)R(Tic)F—NH—CH₂CH₂OCH₃ | | | | | |
| PV-623 (Acid metabolite) | 8.54 | μg/mL | 3.57 | μg/mL | |
| Ac-V(Chg)R(Tic)F—OH | | | | | |

As shown in Table 4, the plasma concentration of PV-3212 is much higher than PV-267 (741 ng/mL vs 275 ng/mL at 0.25 hr; 195 ng/mL vs 91.5 ng/mL at 0.5 hr). In addition, PV-267 is rapidly metabolized to PV-623 (acid metabolite) but PV-3212 is remarkably stable. The ratio of metabolite PV-623 to parent PV-267 at 0.25 hr is 0.67 and 0.73 at 0.5 hr. By contrast, the ratio of PV-623 to PV-3212 is only 0.012 at 0.25 hr and 0.018 at 0.5 hr.

Stabilization of compounds of the series against metabolism is a notable feature of the C-terminal modifications in which the C-terminal amide group is replaced by an amide containing in particular, oligoalkoxyalkyl substituents, particularly variants with 1-10 ethoxy units within the substituent (see Table 5 for examples). These substituents do not compromise biological activity. This is unexpected in view of the decrease in binding for simple alkyl substitution (the N-methyl analog of PV-267 has a binding IC50 of 16 nM vs 2.5 nM for PV-267. The morpholine analog has an IC50 of 33 nM).

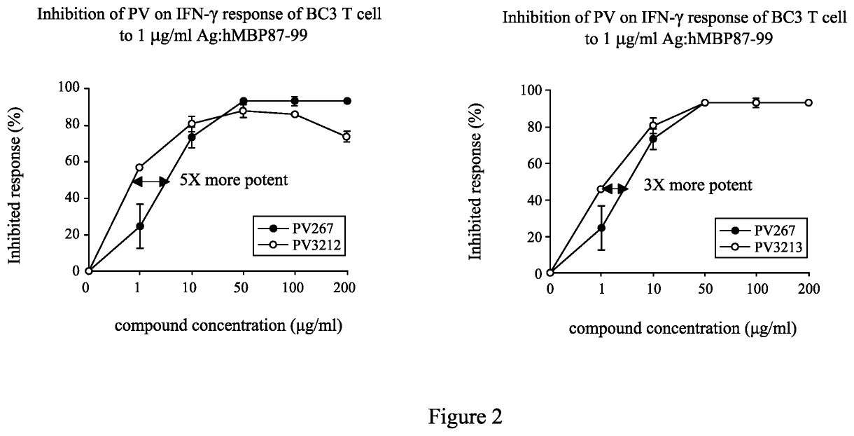

Based on our findings, other analogs with the NH(CH₂CH₂O)ₙ-alkyl (“oligoalkoxyalkylamide”) group substituted for the C-terminal amide group (NH₂) are expected to exhibit potent activity and similar stabilization. For example, PV-3213 which has an NH(CH₂CH₂O)₄CH₃ cap was at least as potent as the unsubstituted PV-267 in the cellular immunology assay as shown in FIG. 2. Representative compounds with this substitution are shown in Table 5.

TABLE 5
Second-generation DRB1*15:01 inhibitor compounds

| PV | |
|---|---|
| Number | Structure (K₁-P₁-P₂-P₃-P₄-P₅-K₂) |
| PV- 03212 | 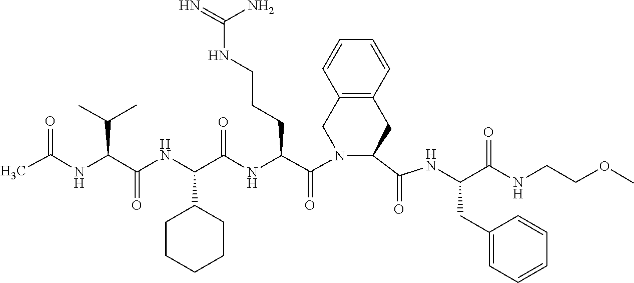 [SMILES: COCCNC(=O)[C@H](Cc1ccccc1)NC(=O)[C@@H]1Cc2ccccc2CN1C(=O)[C@H](CCCNC(=N)N)NC(=O)[C@@H](NC(=O)[C@@H](NC(C)=O)C(C)C)C1CCCCC1] |
| | Ac-V-(Chg)-R-(Tic)-F—NH(CH₂CH₂O)₁CH₃ |
| PV- 03451 | 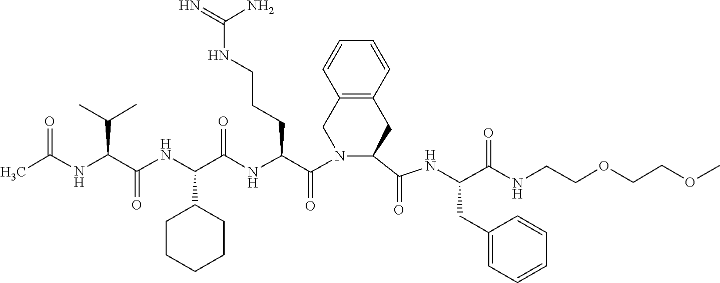 [SMILES: COCCOCCNC(=O)[C@H](Cc1ccccc1)NC(=O)[C@@H]1Cc2ccccc2CN1C(=O)[C@H](CCCNC(=N)N)NC(=O)[C@@H](NC(=O)[C@@H](NC(C)=O)C(C)C)C1CCCCC1] |
| | Ac-V-(Chg)-R-(Tic)-F—NH(CH₂CH₂O)₂CH₃ |
| PV- 03449 | 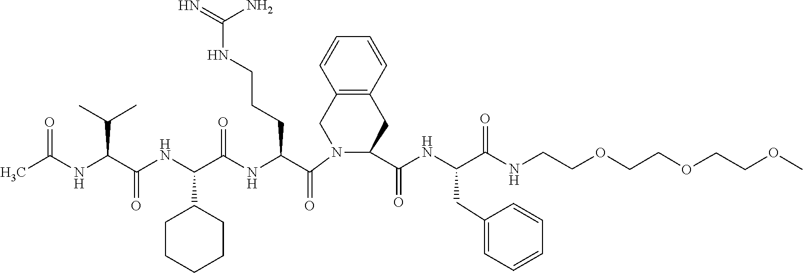 [SMILES: COCCOCCOCCNC(=O)[C@H](Cc1ccccc1)NC(=O)[C@@H]1Cc2ccccc2CN1C(=O)[C@H](CCCNC(=N)N)NC(=O)[C@@H](NC(=O)[C@@H](NC(C)=O)C(C)C)C1CCCCC1] |
| | Ac-V-(Chg)-R-(Tic)-F—NH(CH₂CH₂O)₃CH₃ |
| PV- 03213 | 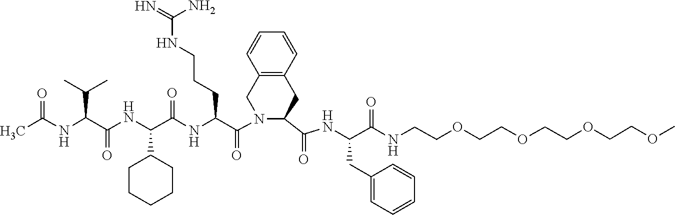 [SMILES: COCCOCCOCCOCCNC(=O)[C@H](Cc1ccccc1)NC(=O)[C@@H]1Cc2ccccc2CN1C(=O)[C@H](CCCNC(=N)N)NC(=O)[C@@H](NC(=O)[C@@H](NC(C)=O)C(C)C)C1CCCCC1] |
| | Ac-V-(Chg)-R-(Tic)-F—NH(CH₂CH₂O)₄CH₃ |

FIG. 2 shows inhibition of IFN-gamma response in the human cellular immunology assay for PV-267, PV-3212 and PV-3213 determined as in FIG. 1 (10,000 BC3 T cells+0.5×10⁶ autologous hPBMCs from DRB1*15:01-positive MS patient (mean±SEM).

Based on these findings, the series of analogs shown in Table 5 were designed to (a) replace the C-terminal primary amide with group K₂ that would maintain the binding characteristics of the one of the amide NH groups at the C-terminus, (b) have a balance of polar and non-polar groups compatible with the hydrophilic region of the MHC DRB1*15:01 binding site (c) retain the complementarity of the residues from P₁ to P₅ with the DRB1*15:01 binding site, and (d) to include a P₁ group to augment binding. These compounds as the C-terminal amides were prepared and characterized. The modification of the C-terminus of the active peptide sequence from a primary amide (K₂=NH₂) to an oligoalkoxylalkylamide moiety (K₂=NH(alkoxy)nalkyl)) was demonstrated to have the desired properties of high potency in the cellular assay and high metabolic stability in vivo.

The unexpected potency in cellular immunology assays coupled with increased metabolic stability of compounds such as the oligoalkoxyalkyl amide-substituted derivatives (e.g., mono-, di-, tri-, and tetra-ethoxymethyl substituted derivatives and other higher homologs) makes these compounds particularly useful in biological studies, determination of stability characteristics in vivo, and in the treatment of autoimmune diseases in patients that carry the DRB1*15:01 gene.

In addition to the compounds with an amino-terminal acyl group (acetyl in PV-267), other modifications of the amino terminus have been shown give compounds with high binding affinity (Table 3). Compounds having K₁ N-capping groups compatible with high affinity binding to DRB1*15:01 thus may be modified at K₂ to include C-capping groups that increase metabolic stability such as oligoalkoxyalkyl amide groups and the like.

Other structural variants at P₁ through P₅ that show good binding affinity may be combined with the newly discovered K₂ groups and/or novel K₁ groups to afford a matrix of representative compounds shown in Table 6.

### EMBODIMENTS OF THE INVENTION

The present invention provides compounds of Formula I (“compound(s) of the invention”), pharmaceutical compositions comprising the compounds of the invention and methods of their use. Without being limited by theory, the compounds of the invention are thought to be inhibitors of antigen presentation by MEW class II HLA-DRB1*15:01 of the formula

K₁—P₁—P₂—P₃—P₄—P₅—K₂ (I)

or a pharmaceutically acceptable salt thereof, wherein:
K₁ is an N-capping group that is alkyl-C(O)-, hydroxyalkyl-C(O)-, aralkyl-C(O)-, heteroarylalkyl-C(O), alkoxy-C(O)-, alkoxycarbonylalkyl-C(O)-, amino-C(O)-, monoalkylamino-C(O)-, dialkylamino-C(O)-, aminoalkyl-C(O)-, monoalkylaminoalkyl-C(O)-, dialkylaminoalkyl-C(O)-, NH₂(CH₂)₄C(O)-, NH₂(CH₂)₃C(O)-, hydroxyalkyl, sulfonyl, aminosulfonyl, alkylaminosulfonyl, dialkylaminosulfonyl, or aryl sulfonyl;
P₁-P₅ are substituents that, when present, are coupled by amide bonds and are each independently amino acyl groups of a natural amino acid or a non-natural amino acid including those set forth in Table 6, below, wherein at least one of P₁-P₅, or at least two of P₁-P₅, or at least three of P₁-P₅ is a non-natural amino acid or peptide mimetic;
K₂ is a substituent, in which the C-terminus is an oligoalkoxyalkyl amide group, i.e., wherein K2 is NH-(alkoxy)ₙ-alkyl.

In one embodiment, the compounds of Formula I contain all L amino acid residues, i.e., (S)-configuration.

In one embodiment, the compounds of Formula I contain one or more residues in the D-, i.e., (R)-configuration.

In one embodiment, the compounds of Formula I are retro-inverso analogs with all of the residues in the D-(R)-configuration with the sequences reversed from C to N.

In one embodiment, the compounds of Formula I contain one or more achiral residues

In one embodiment, K₁, P₁-P₅ and K₂ include any of the groups set forth in Table 6, below. Illustrative non-limiting examples of K₁, P₁-P₅ and K₂ include a contiguous sequence of residues selected from those described in Table 6, below.

In one embodiment, the compounds of Formula I are those set forth in Table 6, below, or a pharmaceutically acceptable salt thereof, which are provided herein by way of illustration and not limitation.

In another embodiment preferred are compounds of the Formula I wherein

K₁ is an N-capping group such as acyl, aminoacyl, heteroacyl or carbamoyl;
K₂ is NH(CH₂CH₂O)ₙ-alkyl wherein n is 1-10;

In another embodiment more preferred are compounds of the Formula I wherein

K₁ is acetyl or aminoacyl and
K₂ is NH—(CH₂CH₂O)ₙCH₃ wherein n is 1-4

In another embodiment most preferred are compounds of the Formula I wherein

K₁ is acetyl and
K₂ is NH—(CH₂CH₂O)ₙCH₃ wherein n is 1-4

In another embodiment most preferred are compounds of the Formula I wherein

K₁ is acetyl and
K₂ is NHCH₂CH₂OCH₃ or NH—(CH₂CH₂O)₄CH₃

In another embodiment, most preferred are compounds of the Formula I wherein

K₁ is aminoacetyl and
K₂ is NH—(CH₂CH₂O)ₙCH₃ wherein n is 1-4.

In another embodiment, compounds of the Formula I comprise the matrix of sequences as described in Table 6, below.

In another embodiment, P1 is Val or aminoacyl; P2 is Cha, Chg, or Idg; P3 is Arg, Cit, or Lys; P4 is

TABLE 6
Representative C- and N-capped sequences: K₁-P₁-P₂-P₃-P₄-P₅-K₂

| N-Cap | [AA] | | | | | C-Cap |
|---|---|---|---|---|---|---|
| K₁ | P₁ | P₂ | P₃ | P₄ | P₅ | K₂ |
| CH₃CO | Val | Chg | Arg | Tic | Phe | NHCH₂CH₂OCH₃ |
| CH₃CH₂CO | t-Bug | Idg | His | Phe | Trp | NH(CH₂CH₂O)₂CH₃ |
| NH₂Gly | Ile | Cpg | Cit | Leu | 4-IndoleAla | NH(CH₂CH₂O)₃CH₃ |
| NH₂Abu | Cpg | Cha | hCit | Ala | 3-BnThienylAla | NH(CH₂CH₂O)₄CH₃ |
| NH₂Sar | Nva | Fua | norCit | Tiq | Phe(4F) | |
| NH₂BetaAla | T(OMe) | Tha | diMeK | Fua | 4-Indazole | |
| Het-acyl | Leu | Val | hArg | Tha | Phe(3,4-diCl) | |
| Carbamoyl | Phg | Phg | norArg | Phg | 1MeTrp | |
| | Thr | Ile | GuanPipGly | Ile | 2Nal | |
| | Chg | Leu | Pyrimidinyl | hPro | 1Nal | |
| | | Trp | 2 Amino His | | Phe(3CN) | |
| | | | (4-Thiaz) Ala | | | |
| | | | Lys | | | |
| | | | Phe | | | |

Abbreviations: NH₂Abu (aminobutyric acid), t-Bug, t-butylglycine; Cpg, cyclopentylglycine; Nva, norvaline; T(OMe); threonine-O-methyl ether; Chg, cyclohexylglycine; Idg, indanylglycine; Phg, phenylglycine; Cit, citrulline; hCit, homocitrulline; norCit, norcitrulline; diMeLysine, N,N-dimethyllysine, hArg, homoarginine; GuanPipGly, guanylpiperidinylglycine

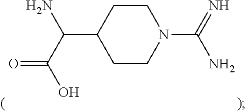 [SMILES: N=C(N)N1CCC(C(N)C(=O)O)CC1]

Pyrimidinyl; pyrimidinylglycine; 2-AminoHis, 2-aminohistidine; (4-Thiaz)A, 4-thiazolylalanine; Tha, thienylalanine; Tic, 1,2,3,4-tetrahydroisoquinoline-3-ylalanine; Tiq, 1,2,3,4-tetrahydroisoquinolin-1-ylalanine; 4-IndolA, 4-indolylalanine; 3-BnThienylA, 3-benzothienylalanine; Phe(4F), 4-flurophenylalanine; 4-Indazole,4-indazolylalanine; Phe(3,4-diCl), 3,4-dichlorophenylalanine; 1MeTrp, 1-methyltryptophan; 2-Nal, 2-naphthylalanine; 1-Nal, 1-naphthylalanine; F(3CN), 3-cyanophenylalanine; [AA], amino acid (see also Table 7).

### Definitions

As used herein, the term “patient” means an animal (e.g., cow, horse, sheep, pig, chicken, turkey, quail, cat, dog, mouse, rat, rabbit, guinea pig), preferably a mammal such as a non-primate and a primate (e.g., monkey and human), most preferably a human. In one embodiment, pre-screening is used to determine whether the patient possesses or is susceptible to having an immune, autoimmune or inflammatory disease or disorder by having a disease-associated MHC class II gene or genes.

As used herein, the term “substituted” means a group substituted by one to four or more substituents, such as, alkyl, halo, trifluoromethyl, trifluoromethoxy, hydroxy, alkoxy, cycloalkyoxy, heterocylooxy, oxo, alkanoyl, aryl, aryloxy, aralkyl, alkanoyloxy, amino, alkylamino, alkylaminoalkyl, alkylamido, arylamino, aralkylamino, cycloalkylamino, heterocycloamino, mono and disubstituted amino in which the two substituents on the amino group are selected from alkyl, aryl, aralkyl, alkanoylamino, aroylamino, aralkanoylamino, substituted alkanoylamino, substituted arylamino, substituted aralkanoylamino, hydroxy, hydroxyalkyl, alkoxyalkyl, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g., SO₂NH₂), substituted sulfonamido, nitro, cyano, carboxy, carbamyl (e.g., CONH2), substituted carbamyl (e.g., CONH alkyl, CONH aryl, CONH aralkyl or instances where there are two substituents on the nitrogen selected from alkyl, aryl or aralkyl), alkoxycarbonyl, aryl, substituted aryl, guanidino and heterocycles, such as, indolyl, imidazolyl, furyl, thienyl, thiazolyl, pyrrolidyl, pyridyl, pyrimidyl and the like. In one embodiment, the substituents are exemplified by the compounds disclosed herein. Wherein, as noted above, the substituents themselves are further substituted, such further substituents are selected from the group consisting of halogen, alkyl, alkoxy, aryl and aralkyl.

As used herein, the term “alkyl” means a saturated straight chain or branched non-cyclic hydrocarbon having from 1 to 20 carbon atoms, preferably 1-10 carbon atoms and most preferably 1-4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl; while saturated branched alkyls include -isopropyl, -sec-butyl, -isobutyl, -tent-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. An alkyl group can be unsubstituted or substituted. Unsaturated alkyl groups include alkenyl groups and alkynyl groups, which are discussed below.

As used herein, the term “alkenyl” means a straight chain or branched non-cyclic hydrocarbon having from 2 to 20 carbon atoms, more preferably 2-10 carbon atoms, most preferably 2-6 carbon atoms, and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl- 1 -butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like. The double bond of an alkenyl group can be unconjugated or conjugated to another unsaturated group. An alkenyl group can be unsubstituted or substituted.

As used herein, the term “alkynyl” means a straight chain or branched non-cyclic hydrocarbon having from 2 to 20 carbon atoms, more preferably 2-10 carbon atoms, most preferably 2-6 carbon atoms, and including at least one carbon-carbon triple bond. Representative straight chain and branched -(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl, and the like. The triple bond of an alkynyl group can be unconjugated or conjugated to another unsaturated group. An alkynyl group can be unsubstituted or substituted.

As used herein, the term “acyl” means an alkanoyl or aroyl group, including acetyl, benzoyl, pivaloyl, cinnamoyl, and the like.

As used herein, the term “halogen” or “halo” means fluorine, chlorine, bromine, or iodine.

As used herein, the term “sulfonamido” means aryl-SONH- or alkyl-SONH, wherein aryl and alkyl are as defined above, including benzenesulfonamido, methanesulfonamido, and the like.

As used herein, the term “alkyl sulfonyl” means —SO₂-alkyl, wherein alkyl is defined as above, including —SO—CH₃, —SO₂—CH₂CH₃, —SO₂—(CH₂)₂CH₃, —SO₂—(CH₂)₃CH₃, —SO₂-(CH₂)₄CH₃, —SO₂—(CH₂)₅CH₃ and the like, and also includes alkyl slufonic acid, including —CH₂—SO₃H, (CH₂)₂—SO₃H, and the like.

As used herein, the term “carboxyl” and “carboxy” mean —COO-. As used herein, the term “alkoxy” means —O-(alkyl), wherein alkyl is defined above, including —OCH₃, —OCH₂CH₃, —O(CH₂)₂CH₃, —O(CH₂)₃CH₃, —O(CH₂)₄CH₃, —O(CH₂)₅CH₃, and the like.

As used herein, the term “alkoxycarbonyl” means —C(═O)O-(alkyl), wherein alkyl is defined above, including —C(═O)O—CH₃, —C(═O)O—CH₂CH₃, —C(═O)O—(CH₂)₂CH₃, —C(═O)O—(CH₂)₃CH₃, —C(═O)O—(CH₂)₄CH₃, —C(═O)O—(CH₂)₅CH₃, and the like. In a preferred embodiment, the esters are biohydrolyzable (i.e., the ester is hydrolyzed to a carboxylic acid in vitro or in vivo).

As used herein, the term “alkoxyalkyl” means -(alkyl)-O-(alkyl), wherein each “alkyl” is independently an alkyl group as defined above, including —CH₂OCH₃, —(CH₂)₂OCH₃, —CH₂OCH₂CH₃, —(CH₂)₂OCH₂CH₃, —(CH₂)₂O(CH₂)₂CH₃, and the like.

As used herein, the term “oligoalkoxyalkyl” means -(alkoxy)nalkyl, wherein each “alkyl” and “alkoxy” group is independently an alkyl group or alkoxy group as defined above, n is 0 to 10, including —CH₂CH₂OCH₃, —(CH₂CH₂O)₂CH₃, —(CH₂CH₂O)₃CH₃, —(CH₂CH₂O)₄CH₃, —(CH₂CH₂O)₂CH₂CH₃, and the like

As used herein, the term “oligoalkoxyalkyl amine” means NH₂-(alkoxyalkyl)ₙ wherein n is 1 to 10 and each “alkoxyalkyl” and “alkyl” group is as defined above, including NH₂CH₂CH₂OCH₃, NH₂(CH₂CH₂O)₂CH₃, NH₂(CH₂CH₂O)₃CH₃, NH₂(CH₂CH₂O)₄CH₃ and the like.

As used herein, the term “oligoalkoxyalkyl amide” means —CONH-(alkoxyalkyl)ₙ wherein n is 1 to 10 and each “alkoxyalkyl” and “alkyl” group is as defined above, including —CONH—CH₂CH₂OCH₃, —CONH—(CH₂CH₂O)₂CH₃, —CONN—(CH₂CH₂O)₃CH₃, —CONN—(CH₂CH₂O)₄CH₃ and the like.

As used herein, the term “aryl” means a carbocyclic aromatic ring containing from 5 to 14 ring atoms. The ring atoms of a carbocyclic aryl group are all carbon atoms. Aryl ring structures include compounds having one or more ring structures such as mono-, bi-, or tricylic compounds as well as benzo-fused carbocyclic moieties such as 5,6,7,8-tetrahydronaphthyl and the like. Preferably, the aryl group is a monocyclic ring, bicyclic ring or tricyclic ring. Representative aryl groups include, but are not limited to, phenyl, tolyl, anthracenyl, fluorenyl, indenyl, azulenyl, phenanthrenyl and naphthyl. A carbocyclic aryl group can be unsubstituted or substituted.

As used herein, the term “heteroatom” means an atom other than carbon, and in a specific embodiment N, O or S.

As used herein, the term “heteroatom group” means a group containing one or more heteroatoms, C and H, including carboxamido, amindino, imino, guanidino, ureido, carbamoyl, and the like.

As used herein, the term “heteroaryl” means an aromatic ring containing from 5 to 14 ring atoms and the ring atoms contain at least one heteroatom, preferably 1 to 3 heteroatoms, independently selected from nitrogen, oxygen, or sulfur. Heteroaryl ring structures include compounds having one or more ring structures such as mono-, bi-, or tricyclic compounds as well as fused heterocyclic moities. Representative heteroaryls are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, benzoquinazolinyl, acridinyl, pyrimidyl, oxetanyl, azepinyl, piperazinyl, morpholinyl, dioxanyl, thietanyl and oxazolyl. A heteroaryl group can be unsubstituted or substituted.

As used herein, the term “carbocyclic” means a carbocyclic ring containing from 5 to 14 ring atoms. The ring atoms of a carbocyclic group are all carbon atoms. Carbocyclic ring structures include compounds having one or more ring structures such as mono-, bi-, or tricylic compounds as well as fused carbocyclic and aryl moieties such a naphthalene, anthracene, indane, indene, phenalene, phenanthrene, benzocyclobutane, benzocycloheptane, tetrahydronaphthalene, and the like. Preferably, the carbocyclic group is a monocyclic ring, bicyclic ring or tricyclic ring. Representative carbocyclic groups include, but are not limited to, cyclopropyl, cyclobutyl, cyclopentyl, cyclohexyl, cycloheptyl, cyclooctyl and the like. A carbocyclic aryl group can be unsubstituted or substituted.

As used herein, the term “heterocyclic” means a ring containing from 5 to 14 ring atoms and the ring atoms contain at least one heteroatom, preferably 1 to 3 heteroatoms per ring, independently selected from nitrogen, oxygen, or sulfur. Heterocyclic ring structures include compounds having one or more ring structures such as mono-, bi-, or trycylic compounds as well as fused heterocyclic moities. Representative heterocyclics include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, imidazolidinyl, isoxazolidinyl, isothiazolidinyl, oxazinanyl, piperazinyl, thiazinanyl, quinolinyl, chromenyl, oxathiolanyl and the like. A heterocyclic group can be unsubstituted or substituted.

As used herein, the term “bicyclic” means a two-ringed system containing from 9-14 carbon atoms wherein one or more, preferably 1-4 or 1-2, of the carbon atoms may be replaced with a heteroatom such as O, N or S. The bicyclic ring system may be saturated, unsaturated, aromatic or non-aromatic. Representative bicyclic rings include, but are not limited to, indole, isoquinoline, quinoline, tetrahydroisoquinoline, and benzofuran.

As used herein, the term “tricyclic” means a three-ringed system containing from 13-17 carbon atoms wherein one or more, preferably 1-6 or 1-3, of the carbon atoms may be replaced with a heteroatom such as O, N or S. The tricyclic ring system may be saturated, unsaturated, aromatic or non-aromatic. Representative tricyclic rings include, but are not limited to, carbazole, phenothiazine, dibenzofuran and fluorene.

As used herein, the term “aryloxy” means —O-aryl group, wherein aryl is as defined above. An aryloxy group can be unsubstituted or substituted.

As used herein, the term “arylalkyl” means -(alkyl)-(aryl), wherein alkyl and aryl are defined above, including —(CH₂)phenyl, —(CH₂)₂phenyl, —(CH₂)₃phenyl, —CH(phenyl)₂, —CH(phenyl)₃, —(CH₂)tolyl, —(CH₂)anthracenyl, —(CH₂)fluorenyl, —(CH₂)indenyl, —(CH₂)azulenyl, —(CH₂)pyridinyl, —(CH₂)naphthyl, and the like.

As used herein, the term “heteroarylalkyl” means -(alkyl)-(heteroaryl), wherein alkyl and heteroaryl are defined above, including —CH₂-triazolyl, —CH₂-tetrazolyl, —CH₂-oxadiazolyl, —CH₂-pyridyl, —CH₂ -furyl, —(CH₂)₂-furyl, —CH₂-benzofuranyl, —CH₂-thiophenyl, —CH₂-benzothiophenyl, —CH₂-quinolinyl, —CH₂-pyrrolyl, —CH₂-indolyl, —CH₂-oxazolyl, —CH₂-benzoxazolyl, —CH₂-imidazolyl, —(CH₂)₂-imidazolyl, —CH₂-benzimidazolyl, —CH₂-thiazolyl, —CH₂-benzothiazolyl, —CH₂-isoxazolyl, —CH₂ -pyrazolyl, —CH₂-isothiazolyl, —CH₂-pyridazinyl, —CH₂-pyrimidinyl, —CH₂-pyrazinyl, —CH₂-triazinyl, —CH₂-cinnolinyl, —CH₂-phthalazinyl, —CH₂-quinazolinyl, —CH₂-pyrimidyl, —CH₂-oxetanyl, —CH₂-azepinyl, —CH₂-piperazinyl, —CH₂-morpholinyl, —CH₂-dioxanyl, —CH₂-thietanyl, —CH₂-oxazolyl, —(CH₂)2-triazolyl, and the like.

As used herein, the term “heteroalkyl” means an alkyl group, as defined above, wherein one or more of the —CH₂- groups is replaced with a heteroatom independently selected from nitrogen, oxygen, or sulfur, including ether, thioether and alkylamino groups such as —CH₂—O—CH₃, —CH₂—S—CH₃, —CH₂—NH—CH₃, —CH₂—O—CH₂—CH₃, —CH₂—S—CH₂—CH₃, —CH₂—NH—CH₂—CH₃, —CH₂—O—(CH₂)₂—CH₃, —CH₂—S—(CH₂)₂—CH₃, —CH₂—NH—(CH₂)₂-CH₃, —CH₂—O—(CH₂)₃—CH₃, —CH₂—S—(CH₂)₃—CH₃, —CH₂—NH—(CH₂)₃—CH₃ including guanidino, amidino and the like.

As used herein, the term “hydroxyalkyl” means alkyl, wherein alkyl is as defined above, having one or more hydrogen atoms replaced with hydroxy, including —CH₂OH, —CH₂CH₂OH, —(CH₂)₂CH₂OH, —(CH₂)₃CH₂OH, —(CH₂)₄CH₂OH, —(CH₂)₅CH₂OH, —CH(OH)—CH₃, —CH₂CH(OH)CH₃, and the like.

As used herein, the term “hydroxy” means —OH. As used herein, the term “oxoarylalkyl” means —O-(alkyl)-(aryl), wherein alkyl and aryl are defined above, including —O—(CH₂)₂phenyl, —O—(CH₂)₃phenyl, —O—CH(phenyl)₂, —O—CH(phenyl)₃, —O—(CH₂)tolyl, —O—(CH₂)anthracenyl, —O—(CH₂)fluorenyl, —O—(CH₂)indenyl, —O—(CH₂)azulenyl, —O—(CH₂)pyridinyl, —O—(CH₂)naphthyl, and the like.

As used herein, the term “cycloalkyloxy” means —O-(cycloalkyl), wherein cycloalkyl is defined above.

As used herein, the term “cycloalkylalkyloxy” means —O-(alkyl)-(cycloalkyl), wherein cycloalkyl and alkyl are defined above, including —O-methylcyclopropyl, —O-methylcyclobutyl, —O-1-ethyl-2-cyclohexyl, and the like.

As used herein, the term “aminoalkoxy” means —O-(alkyl)-NH₂, wherein alkyl is defined above, including —O—CH₂—NH₂, —O-(CH₂)₂—NH₂, —O—(CH₂)₃—NH₂, —O—(CH₂)₄-NH₂, —O—(CH₂)₅—NH₂, and the like.

As used herein, the term “alkylamino” means —NH-(alkyl) or —N-(alkyl)(alkyl), wherein alkyl is defined above, including —NH—CH₃, —NH—CH₂CH₃, —NH—(CH₂)₂CH₃, —NH—(CH₂)₃CH₃, —NH—(CH₂)₄CH₃, —NH—(CH₂)₅CH₃, —N—(CH₃)₂, —N—(CH₂CH₃)₂, —N—((CH₂)₂CH₃)₂, —N—(CH₃)(CH₂CH₃), and the like.

As used herein, the term “alkylamido” means -(alkyl)-NH—C(O)(alkyl), wherein each “alkyl” is independently an alkyl group defined above including —CH₂—NH—C(O)CH₃, —CH₂—NH—C(O)CH₂CH₃, —CH₂—NH—C(O)(CH₂)₂CH₃, —CH₂—NH—C(O)(CH₂)₃CH₃, —CH₂—NH—C(O)(CH₂)₄CH₃, —CH₂—NH—C(O)(CH₂)₅CH₃, —(CH₂)₂—NH—C(O)CH₃, —(CH₂)₂—NH—C(O)CH₂CH₃, —(CH₂)₂—NH—C(O)(CH₂)₂CH₃, and the like or -(alkyl)-C(O)-NH-(alkyl), wherein each “alkyl” is independently an alkyl group defined above including —CH₂—C(O)—NH—CH₃, —CH₂—C(O)—NH—CH₂CH₃, —CH₂—C(O)—NH—(CH₂)₂CH₃, —CH₂—C(O)—NH—(CH₂)₃CH₃, —CH₂—C(O)—NH—(CH₂)4CH₃, —CH₂—C(O)—NH—(CH₂)₅CH₃, —(CH₂)₂—C(O)—NH—CH₃, —(CH₂)₂—C(O)—NH—CH₂CH₃, —(CH₂)₂—C(O)—NH—(CH₂)₂CH₃, and the like.

As used herein, the term “dialkylaminoalkyl” means -(alkyl)-N(alkyl)(alkyl), wherein each “alkyl” is independently an alkyl group defined above, including —CH₂—N(CH₃)₂, —CH₂—N(CH₂CH₃)₂, —CH₂—N((CH₂)₂CH₃)₂, —CH₂—N(CH₃)(CH₂CH₃), —(CH₂)₂—N(CH₃)₂, and the like.

As used herein, the term “arylamino” means —NH(aryl), wherein aryl is defined above, including —NH(phenyl), —NH(tolyl), —NH(anthracenyl), —NH(fluorenyl), —NH(indenyl), —-NH(azulenyl), —NH(pyridinyl), —NH(naphthyl), and the like.

As used herein, the term “arylalkylamino” means —NH-(alkyl)-(aryl), wherein alkyl and aryl are defined above, including —NH-CH₂-(phenyl), —NH—CH₂-(tolyl), —NH—CH₂-(anthracenyl), —NH—CH₂-(fluorenyl), —NH—CH₂-(indenyl), —NH—CH₂-(azulenyl), —NH—CH₂-(pyridinyl), —NH—CH₂-(naphthyl), —NH—(CH₂)₂-(phenyl) and the like.

As used herein, the term “cycloalkylamino” means -NH-(cycloalkyl), wherein cycloalkyl is defined above, including —NH-cyclopropyl, —NH-cyclobutyl, —NH-cyclopentyl, —NH-cyclohexyl, —NH-cycloheptyl, and the like.

As used herein, the term “aminoalkyl” means -(alkyl)-NH₂, wherein each “alkyl” is independently an alkyl group defined above, including —CH₂—NH₂, —(CH₂)₂, —NH₂, —(CH₂)₃—NH₂, —(CH₂)₄—NH₂, —(CH₂)₅—NH₂ and the like.

As used herein, the term “alkylaminoalkyl” means -(alkyl)-NH(alkyl) or -(alkyl)-N(alkyl)(alkyl), wherein each “alkyl” is independently an alkyl group defined above, including —CH₂—NH—CH₃, —CH₂—NHCH₂CH₃, —CH₂—NH(CH₂)₂CH₃, —CH₂—NH(CH₂)₃CH₃, —CH₂—NH(CH₂)₄CH₃, —CH₂—NH(CH₂)₅CH₃, —(CH₂)₂—NH—CH₃, —CH₂—N(CH₃)₂, —CH₂—N(CH₂CH₃)₂, —CH₂—N((CH₂)₂CH₃)₂, —CH₂—N(CH₃)(CH₂CH₃), —(CH₂)₂—N(CH₃)₂, and the like.

As used herein, the term “alkoxyaminoalkyl” means —O-alkyl-NH(alkyl) or —O-alkyl-N(alkyl)(alkyl), where alkyl and aminoalkyl are as defined above, including —OCH₂CH₂N(CH₃)₂, and the like.

As used herein, the term “N-cap” means a moiety attached to the N-terminal residue of the compound including acetyl, (alkyl)carbamoyl, aminoacyl, and the like.

As used herein, the term “C-cap” means a moiety attached to the C-terminal residue of the compound, including NH₂ and NH-(oligoalkoxy)alkyl, NHCH₂CH₂OCH₃, NH(CH₂CH₂O)₂CH₃, NH(CH₂CH₂O)₃CH₃, NH(CH₂CH₂O)₄CH₃, and the like.

As used herein, the term “sugar moiety” means monosaccharides (e.g., glucose, arabinose, fucose, galactose, mannose, xylose, fructose, lyxose, allose, arinose, ribose, talose, gulose, idose, altrose, sorbitol, mannitol or glucosamine), disaccharides and oligosaccharides (e.g., maltose, isomaltose, turanose, gentiobiose, melibiose, planteobiose, primererose, vicianose, nigerose, laminaribiose, rutinose, cellobiose, xylobiose, maltotriose, gentianose, melezitose, planteose, ketose, trehalose, sucrose, lactose, raffinose or xylotriose), polysaccharides (e.g., amylose, ficol, dextrin, starch, dextran, polydextrose, pullulan, cyclodextrin, glucomannoglycan, glucomannan, guar gum, gum arabic or glycosaminoglycan), complex carbohydrates (e.g., glycopeptide, glycoprotein, glycolipid or proteoglycan), and the like.

As used herein, the term “PEG” means a polyethylene glycol group such as H(OCH₂CH₂)ₙOH, wherein n is 1-30, 1-25, 1-20, 1-15, 1-10, 1-5 or 1-2.

As used herein, a “therapeutically effective amount” refers to that amount of the compound described herein or other active ingredient sufficient to provide a therapeutic benefit in the treatment or management of the disease (e.g., a genetic disease, a central nervous system (“CNS”) disease, an inflammatory disease, a neurodegenerative disease or an autoimmune disease) or to delay or minimize symptoms associated with the disease or to prevent or delay progression of a disease. Further, a therapeutically effective amount with respect to a compound described herein means that amount of therapeutic agent alone, or in combination with other therapies, that provides a therapeutic benefit in the treatment or management of the disease. Used in connection with an amount of a compound described herein, the term can encompass an amount that improves overall therapy, reduces or avoids symptoms or causes of disease, or enhances the therapeutic efficacy of or synergies with another therapeutic agent.

As used herein, a “prophylactically effective amount” refers to that amount of a compound described herein or other active ingredient sufficient to result in the prevention, recurrence or spread of the disease (e.g., a genetic disease, a CNS disease, an inflammatory disease, a neurodegenerative disease or an autoimmune disease). A prophylactically effective amount may refer to the amount sufficient to prevent initial disease or the recurrence or spread of the disease or the occurrence of the disease in a patient, including but not limited to those predisposed to the disease. A prophylactically effective amount may also refer to the amount that provides a prophylactic benefit in the prevention of the disease. Further, a prophylactically effective amount with respect to a compound described herein means that amount alone, or in combination with other agents, that provides a prophylactic benefit in the prevention of the disease. Used in connection with an amount of a compound described herein, the term can encompass an amount that improves overall prophylaxis or enhances the prophylactic efficacy of or synergies with another prophylactic agent.

As used herein, a “therapeutic protocol” refers to a regimen of timing and dosing of one or more therapeutic agents.

As used herein, a “prophylactic protocol” refers to a regimen of timing and dosing of one or more prophylactic agents.

A used herein, a “protocol” includes dosing schedules and dosing regimens.

As used herein, “in combination” refers to the use of more than one prophylactic and/or therapeutic agent on a patient in a manner such that the patient benefits from both drugs. The drugs may be administered simultaneously or sequentially. In one embodiment, the compound described herein and the other prophylactic or therapeutic agent exert their biological effect on the patient during the same time period.

As used herein, the terms “prevent”, “ preventing” and “prevention” refer to the prevention of the onset, recurrence, or spread of the disease (e.g., a genetic disease, a CNS disease, an inflammatory disease, a neurodegenerative disease or an autoimmune disease) in a patient. In one embodiment, the patient shows signs of an autoimmune disease, particularly multiple sclerosis, or has a first lesion, and administration of a compound described herein prevents worsening of the symptoms or the formation of additional lesions or the transition of an early form of multiple sclerosis (such as relapsing-remitting multiple sclerosis) to secondary progressive multiple sclerosis.

As used herein, the terms “treat”, “treating” and “treatment” refer to the eradication or amelioration of the disease (e.g., a genetic disease, a CNS disease, an inflammatory disease, a neurodegenerative disease or an autoimmune disease) or symptoms associated with the disease or to the management of the disease which does not result in a cure of the disease or reduction of the disease but prevents its progression. In certain embodiments, such terms refer to minimizing the spread or worsening of the disease resulting from the administration of one or compounds described herein to a patient with such a disease. In one embodiment, a patient is administered one or more compounds described herein to manage a disease so as to prevent the progression or worsening of the disease.

As used herein, the term “pharmaceutically acceptable salts” refer to salts prepared from pharmaceutically acceptable non-toxic acids or bases including inorganic acids and bases and organic acids and bases. Suitable pharmaceutically acceptable base addition salts for the compounds described herein include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, trifluoroacetic acid and p-toluenesulfonic acid. Specific non-toxic acids include acetic, hydrochloric, hydrobromic, phosphoric, sulfuric, trifluoracetic, and methanesulfonic acids. Examples of specific salts thus include hydrochloride, trifluoroacetate, and acetate salts.

As used herein, the term “prodrug” means a derivative of a compound that can hydrolyze, oxidize, or otherwise react under biological conditions (in vitro or in vivo) to provide an active compound, particularly a compound described herein. Examples of prodrugs include, but are not limited to, derivatives and metabolites of a compound described herein that include biohydrolyzable moieties such as biohydrolyzable amides, biohydrolyzable esters, biohydrolyzable carbamates, biohydrolyzable carbonates, biohydrolyzable ureides, biohydrolyzable lipids and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers Gmfh).

As used herein, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, alkoxyacyloxy esters, alkyl acylamino alkyl esters, and choline esters. Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, the term “optically pure” or “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein, the term “enantiomerically pure” means a stereomerically pure composition of a compound having one or more chiral centers.

As used herein, the term “compound described herein ” means a compound described herein which is capable of inhibiting antigen binding to an MHC class II HLA-DRB1*15:01 molecule and thus inhibiting T cell proliferation in vitro or in vivo. Such inhibitory activity can be determined by an assay or animal model well-known in the art including those set forth in herein. The compound described herein can be in the form of a pharmaceutically acceptable prodrug, salt, solvate or hydrate thereof.

As used herein, the compounds described herein may optionally also be “capped” at the N-terminus wherein the N-cap is a group such as an acyl group, an aminoacyl group, a sulfonyl group, a carbamate, or a heteroacyl or heterosulfonyl group that is covalently attached to the N-terminal amino group.

The compounds described herein are capped at the C-terminus where the C-capping group is covalently attached to the C-terminal carboxyl group, replacing the COOH group with an ester, carbamate, or amide group. The C-capping group may be an amino group (forming a C-terminal amide) or a substituted amino group substituent may include an alkoxyalkyl group or an oligoalkoxyalky group. Examples of C-capping groups include, but are not limited to, NH₂, NH—CH₃, NHCH₂CH₂OCH₃, NH(CH₂CH₂O)₂CH₃, NH(CH₂CH₂O)₃ CH₃, NH(CH₂CH₂O)₄CH₃ and —NR₂ (wherein each occurrence of R is independently H, alkyl, alkoxyalkyl, or oligoalkoxyalkyl).

The compounds described herein may contain both N- and C-capping groups, including compounds including sequences described in Tables 5 and 6. Further examples of capping groups include those disclosed in U.S. Pat. No. 6,020,315, issued Feb. 1, 2000, incorporated by reference herein in its entirety. In a particular embodiment, the compound described herein is a compound of the Formula I. In another particular embodiment, the compound described herein is one of compounds in Table 6. In another particular embodiment, the compound described herein is one of the compounds in Table 5.

As used herein, the terms “naturally occurring amino acid” or “natural amino acid” refer to any of the 20 naturally occurring L-amino acids (S-configuration) as set forth in Table 7, below, and also include mixtures which contain about 0.1%, about 0.5%, about 1%, about 5%, about 10%, about 25%, about 50%, about 75%, about 90%, about 95%, about 99%, about 99.5% or about 99.9% by weight of the corresponding D-amino (R-configuration) acid. The amino acids can be used to prepare or are a part of the compounds described herein. The linkage between each amino acid of the compounds described herein may be an amide, a substituted amide or an isostere of amide.

TABLE 7
Abbreviations for natural L-amino acids

| | Three-letter | One-letter |
|---|---|---|
| Amino acid | abbreviation | symbol |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gln | Q |
| Glutamic acid | Glu | E |
| Glutamine or glutamic acid | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the terms “non-naturally occurring amino acid” or “non-natural amino acid” refers to any natural amino acid that has been modified or any structure having amino and carboxyl groups (e.g., ornithine), including peptide mimetics encompassing more than one amino acid in length, or non-peptide peptide mimetics having backbone replacements for the peptide backbone, represented by P₁, P₂, P₃, P₄, or P₅, below.

As used herein, non-classical amino acids or chemical amino acid analogues are also used to prepare or are a part of the compounds described herein. Non-classical amino acids include, but are not limited to, the D-isomers (R-configuration) of the common amino acids, α-O-amino isobutyric acid, 4-aminobutyric acid, Abu, 2-amino butyric acid, (-Abu, -Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-O-alanine, fluoro-amino acids, designer amino acids such as β-O-methyl amino acids, Cα-O-methyl amino acids, Nα-O-methyl amino acids, cyclohexylglycine, cyclopentylglycine, Tic (1,2,3,4-tetrahydro-isoquinoline-3-carboxylic acid, Tiq, (1,2,3,4-tetrahydro-isoquinoline-1-carboxylic acid, dipeptide mimetics such as Haic (2S,5S)-5-amino-1,2,4,5,6,7-hexahydro-4-oxo-azepino[3,2,1-h]indole-2-carboxylic acid) and other amino acid analogues described in Table 6 in general. Furthermore, the amino acid or non-classical amino acids or analogues can have R- or S-configurations; S-is preferred.

More specifically, provided herein are compounds comprised of either enantiomer of an amino acid, non-natural amino acid, non-classical amino acid or chemical amino acid analogue. With respect to the use of non-natural amino acids, non-classical amino acids and chemical amino acid analogues, the stereochemical variation is robust and provided herein are compounds comprising one or more enantiomers or epimers at one or more of the K₁, K₂ or P₁-P₅ positions. Also provided herein are retro-inverso compounds in which all the amino acids are of the R configuration but the sequence is reversed from N to C to C to N.

It should be noted that if the stereochemistry of a structure or a portion of a structure is not indicated with, for example, bold or dashed lines, the structure or portion of the structure is to be interpreted as encompassing all stereoisomers of it.

Compounds in Table 6 can be assayed using the assay protocol set forth herein. Preferred compounds of the invention are those with IC₅₀ values of less than about 1 less than about 500 nM, less than about 250 nM, less than about 100 nM, less than about 50 nM, less than about 25 nM, less than about 10 nM, less than about 5 nM or less than about 1 nM using the assay protocol set forth herein.

In one embodiment, the compounds of Table 6 contain all L residues ((5)-configuration).

In one embodiment, the compounds of Table 6 contain one or more residues in the D ((R))-configuration.

Also included within the scope of the present invention are “capped” forms of the compounds of the invention, i.e., N-capped or C-capped forms. N-capped forms (K₁) include a moiety capable of reacting with the N-terminal —NH₂. In one embodiment, N-terminal blocking groups include RC(O)-, where R is —H, (C₁₋₆) alkyl, (C-₁₋₆) alkenyl, (C₁₋₆) alkynyl, (C₅₋₂₀) aryl, (C₆₋₂₆) alkaryl, 5-20 membered heteroaryl, aminoalkyl, or 6-26 membered alkylheteroaryl. In another embodiment, the N-terminal blocking group may be a sulfonyl group, such as C1-C6 alkyl sulfonyl or heteroaryl sulfonyl, and the like. In a particular embodiment, N-terminal blocking groups include acetyl and aminoacyl. In one embodiment, C-terminal blocking groups (K₂) include oligoalkoxyalkyl. In a particular embodiment, C-terminal blocking groups have the NH(CH₂CH₂O),-alkyl group wherein n is 1-10 attached to the C-terminal carboxyl group.

Preferred compounds of the invention are compounds of Formula I that are resistant to cathepsin, particularly cathepsin B, D or L, degradation and are not metabolized in vivo to the C-terminal acid. In a preferred embodiment, the compounds of Formula I have a half-life of greater than about 1 hour in a solution comprising cathepsin B, preferably greater than about 2 hours, more preferably greater than about 3 hours and most preferably greater than about 4 hours.

In another embodiment, the compounds of the invention, such as the compounds of Formula I, are resistant to degradation by peptidases in vitro.

In another embodiment, the compounds of the invention, such as the compounds of Formula I, are resistant to degradation in a cellular environment.

In another embodiment, the compounds of the invention, such as the compounds of Formula I, are resistant to degradation by peptidases in vivo.

In another embodiment, the compounds of the invention, such as the compounds of Formula I, are resistant to metabolism in vitro or in vivo that would transform the K₂ group to the peptide acid (K₂═OH).

In another preferred embodiment, the compounds of the invention, including, but not limited to, the compounds of Formula I, bind preferentially to MEW class II HLA-DRB1*15:01 and do not bind with at least a 5-fold selectivity to other MHC class II molecules. In a particular embodiment, the compounds of the invention, including, but not limited to, the compounds of Formula I, have IC₅₀ values for an HLA DRB1*15:01 molecule which are about 0.5, about 0.1, about 0.01, about 0.001 or about 0.0001 of their IC₅₀ value for another HLA-DR, HLA-DP or HLA-DQ molecule.

Without being limited by any theory, in one embodiment the compounds of the invention, including, but not limited to, the compounds of Formula I, competitively inhibit the binding of autoantigens to MEW class II HLA-DRB1*15:01 and block antigen presentation to autoreactive T-cells, inhibiting autoreactive T cell proliferation, cytokine production, and resulting pathology.

### Biological Assays

Without being limited by theory, the principle of antigen presentation is thought to require antigen binding by MEW class II molecules. In the example of multiple sclerosis, the MEW class II molecule, HLA-DRB1*15:01 presents autoantigens derived from myelin that initiate T cell activation and destruction of the myelin insulation of nerves. Accordingly, the binding affinity of the compounds of the invention to MHC class II HLA-DRB1*15:01 molecules is one indicator of their usefulness as therapeutics for the treatment of an autoimmune disease. More specifically, inhibition of binding of MEW class II HLA-DRB1*15:01 is an indicator of usefulness as a therapeutic for multiple sclerosis, Goodpasture's disease or other DRB1*15:01 associated autoimmune diseases.

Assays useful for demonstrating the usefulness the compounds of the invention include those HLA molecule binding assays known in the art, such as Texier, C. et al. (2000) J. Immunol. 164:3177-3184; Jones, A. et al. (1999) Bioorg. Med. Chem. Lett. 9:2115-2118; Jones, A. et al. (1999) Bioorg. Med. Chem. Lett. 9:2109-2114; Wucherpfennig et al. (1994) J. Exp. Med. 179:279-290; and Bolin, D. R. et al. (2000) J. Med. Chem. 43:2135-2148, each of which is incorporated by reference herein in its entirety.

Assays useful for demonstrating the T cell proliferation inhibitory activity of the compounds of the invention include the assay set forth herein as well as those assays known in the art, such as Sarabu, R. (2002) Drug. Des. Disc. 18:3-7; Bolin, D., (2000) J. Med. Chem. 43:2135-2148; Chirathaworn, C. (2002) J. Immunol. 168(11):5530-5537; Falcioni, F., et. al. (1999) Nature Biotechnology 17:562-567; Ji, et al. (2013) J. Immunol. 191:5074-84, each of which is incorporated by reference herein in its entirety.

Animal models useful for demonstrating the therapeutic utility of the compounds of the invention include those known in the art, such as Rosloniec, E F, (1998), J. Immunol. 160(6), 2573-8, and Rosloniec, E F, (2003), Springer Semin. Immunopathol., (1), 3-18 , and Ji, et al. (2013) J. Immunol. 191:5074-84, each of which is incorporated by reference herein in its entirety.

Assays useful for demonstrating stability of the compounds of the invention to cathepsin degradation include those known in the art, such as Li, M. (1993) Bioconjug. Chem. 4:275-83 and Nakagomi, K. (2002) Biol. Pharm. Bull. 25:564-8.

Extensive methodology and assays used to characterize inhibitors of DRB 1 *15:01 are described in the comprehensive publications of Ji, et al., J Immunol. 2013 Nov 15;191(10):5074-84 and Huynh M., et al., J Autoimmun. 2019 103:102276.

### Pharmacokinetics and Metabolism

The pharmacokinetics/metabolism study was performed in non-fasted C57BL/6J mice. Dose formulation was prepared on the day of treatment, using 10% v/v DMSO solution and 90% sterile water for injection as vehicle. Blood samples were collected at 0.25, 0.5, 1, 2, 4, 6, 8, 12 and 24 hr post-dose (n=9 mice/group, staggered sampling with 3 mice/time point). At each time point, under isoflurane anesthesia approximately 70 μL of blood was collected through retro orbital plexus puncture and transferred into a pre-labeled tube. 50 μL of blood was taken from the prelabeled tube and it was added into a labelled tube containing 200 μL of acetonitrile to precipitate proteins. Immediately, blood samples were vortexed to facilitate mixing of acetonitrile with the blood. Samples were kept on dry ice at −70° C. until analysis.

Assay Protocol

| | | | Dose | |
|---|---|---|---|---|
| Group | Treatment | Dose | Volume | Number of Mice |
| 1 | PV-3212 | 10 mg/kg, IP | 4 mL/kg | (n = 9 mice/group, |
| | | | | staggered sampling with |
| | | | | 3 time points/mouse) |
| 2 | PV-267 | 10 mg/kg, IP | 4 mL/kg | (n = 9 mice/group, |
| | | | | staggered sampling with |
| | | | | 3 time points/mouse) |

Bioanalysis was performed using fit-for-purpose analytical method using LC-MS/MS method for the quantification of PV-267 (Parent) or PV-3212 (Parent) and PV-623 (Metabolite) in mice blood samples and the linearity range was 2 to 2500 ng/mL for PV-267 (Parent), PV-3212 (Parent) and PV-623 (metabolite).

The blood pharmacokinetic parameters for PV-267 and PV-3212 were calculated by using standard non-compartmental analysis (Phoenix software, version 6.4, Pharsight Corporation, Mountain View, Calif. 94040/USA) using the sparse sampling model and linear trapezoidal with linear interpolation calculation method. Pharmacokinetic Parameters for PV-623 (metabolite) were not calculated due to insufficient blood concentrations.

Following PV-267 single intraperitoneal administration to C57BL/6J mice at a target dose of 10 mg/kg, Parent (PV-267) peak blood concentration of 275 ng/mL and Metabolite (PV-623) peak blood concentration of 169 ng/mL was observed at 0.25 hr post dose.

Mean metabolite (PV-623) to parent (PV-267) ratio was found to be 0.61 at 0.25 hr and 0.73 at 0.50 hr post dose. Thus the formation of metabolite (i.e., PV-623) was high (Cmax =169 ng/mL). The data are shown in Table 4, above.

Following PV-03212 single intraperitoneal administration to C57BL/6J mice at a target dose of 10 mg/kg, Parent (PV-03212) peak blood concentration of 741 ng/mL was observed at 0.25 hr

### Synthesis and Preparation of Embodiments

The compounds of the invention can generally be prepared via solid-phase synthesis procedures such as those described in Barany, G. and Merrifield, R. B. The Peptides, Gross E., Meienhofer, J. Eds., Academic Press: New York, 1980, vol. 2, pp. 1-284; Solid phase synthesis: A practical guide, S. A. Kates, F. Albericio, Eds. Marcel Dekker: New York, 2000; Myers A. G. et al. (1997) J. Amer. Chem. Soc. 119:656; Myers A. G. et al. (1999) J. Org. Chem. 64:3322D; A. Wellings, E. Atherton, (1997) Methods Enzymol. 289:44; Fields, G. B. et al., (1990) Int. J. Peptide Protein Res. 35:161; H. Rink, (1987) Tetrahedron Lett. 28: 3787; R. C. Sheppard, B. J. Williams, (1982) Int. J. Rept. Protein Res. 20:451; J. Coste, et al., (1991) Tetrahedron Lett. 32:1967; L. A. Carpino, A. Elfaham, C. A. Minor, F. Albericio, (1994) J. Chem. Soc. Chem. Comm., 201; M. Felix, et al., (1998) J. Peptide Res. 52:155; U.S. Pat. No. 5,770,732 issued Jun. 23, 1998; U.S. Pat. No. 5,514,814 issued May 7, 1996; and U.S. Pat. No. 5,489,692 issued Feb. 6, 1996, which are incorporated by reference herein in their entirety.

Alternatives include solution-phase fragment coupling of protected amino acids and dipeptides. (Tsuda, Y., et al., (2001) Solution-Phase Peptide Synthesis, in “Amino Acids, Peptides and Proteins in Organic Chemistry: Building Blocks, Catalysis and Coupling Chemistry, Volume 3, Wiley, Hughes, Ed., (2011) 201-251).

Starting materials useful for preparing the compounds of the invention and intermediates therefore, are commercially available or can be prepared from commercially available materials using known synthetic methods and reagents.

In general, amino acids (natural, non-natural, or peptide mimetics) are protected as N-Fmoc derivatives with acid-labile protecting groups as appropriate on reactive side chain substituents. Fmoc-Rink or Knorr linker-BHA resin is used for the synthesis of C-terminal amides. TGT-alcohol resin or 2-chlorotrityl resin is used for the synthesis of C-terminal acids. Fmoc groups are removed using 20-40% piperidine in DMF. Condensation of the appropriate N-Fmoc amino acid is accomplished using HBTU/N-methyl morpholine in DMF (the HOBT active ester of the amino acid is preformed and added to the resin). Couplings to N-alkyl or imino acids are performed with either BOP-Cl or PyBrOP in NMP. After deprotection of the Fmoc group using 20-40% piperidine in DMF, coupling of the next N-Fmoc amino acid or capping group is accomplished in the same manner. This cycle is repeated until the desired sequence has been synthesized on the resin.

Final deprotection of the N-terminal Fmoc group, if present, is followed by treatment with an acid anhydride, activated carboxylic acid or sulfonic acid in DMF for 1 hour to give N0-capped compounds. When C-terminal amides are desired, the resin is washed with DMF, ethanol, methylene chloride and dried in vacuo. The linear compounds are cleaved from the resin and any side chain protecting groups are removed by treatment with a 80% solution of TFA in dichloromethane, with the addition of water (5%) and triisopropylsilane (5%). Filtrates are concentrated in vacuo and diluted with diethyl ether to afford crude compounds as white solids. Crude products are purified by reverse phase HPLC (C18 silica gel; acetonitrile/water/TFA gradient elution) and lyophilized to give the final compounds. When the sequence includes a basic substituent, such as an amino group of a lysine, the product may be formed as a TFA salt. If desired, the TFA salt can be exchanged for another pharmaceutically acceptable salt by neutralization and treatment with a pharmaceutically acceptable acid to form a new salt.

The final products can be characterized by analytical HPLC, FAB-MS, ES-MS and/or amino acid analysis. HPLC purity, as determined from all UV active peaks, is typically greater than 97%.

Non-natural, non-alpha amino acids and peptide mimetics are incorporated into sequences by the same methodology and, if required, the couplings are followed by detection of any unreacted free amino terminus using a standard Kaiser test. In such instances, couplings are repeated until a negative Kaiser test is obtained.

When C-terminal groups other than amide are desired, the synthesis is carried out on TGT-alcohol resin or 2-chlorotrityl resin as described above, except that the side chain-protected compound is cleaved from the resin with 2% TFA to release a protected compound carboxylic acid, which can in a separate step be amidated or converted to a C-capped derivative as in compounds of the Formula I, and subsequently deprotected as described above. In cases where simultaneous side chain and resin cleavage at the same time is desired, then 95% TFA may be used to generate the C-terminal acid of the deprotected peptide.

Alternately, when C-capped sequences of the Formula I are desired, a compound in which the C-terminus is NH2 may be hydrolyzed by treatment with 6N HCl to convert it to the corresponding acid where the C-terminus is OH. The acid may then be coupled with an amine to generate the C-capped sequence of Formula I. In the case of compounds in which the P3 group is Arg, it is not necessary to protect the Arg side chain before coupling with the C-cap amine.

For example, the compounds in Table 5 were prepared by coupling the acid PV-623 with the appropriate oligoalkylamine using HOAt (1-hydroxy-7-azabenzotriazole), TMP (2,4,6-trimethylpyridine), and DIC (N,N′-diisopropylcarbodiimide) in dichloromethane at room temperature overnight. The mixture was evaporated and purified by prep HPLC eluting with 0.075% TFA in acetonitrile-water to give the compounds shown in Table 5.as their TFA salts.

### Methods of Use

A first embodiment of the invention relates to a method for inhibiting antigen binding to an MEW class II molecule in vitro or in vivo, particularly an MHC class II HLA-DRB1*15:01 molecule, comprising contacting a cell, such as a mammalian cell, with an effective amount of a compound of the invention. In a preferred embodiment, the MHC class II molecule is HLA-DRB1*15:01. In one embodiment, the compound of the invention competitively inhibits an antigen associated with an autoimmune disease from binding to the MHC class II HLA-DRB1*15:01 molecule.

In another embodiment, the invention relates to a method for inhibiting antigen presentation by an MHC class II HLA-DRB1*15:01 molecule in vitro or in vivo, comprising contacting a cell, such as a mammalian cell, with an effective amount of a compound of the invention. In a preferred embodiment, the MEW class II molecule is MHC class II HLA-DRB1*15:01.

In another embodiment, the invention relates to a method for inhibiting T cell proliferation in vitro or in vivo comprising contacting a cell, such as a mammalian cell, with an effective amount of a compound of the invention.

In another embodiment, the invention relates to a method for treating or preventing a disease treatable or preventable by inhibiting T cell proliferation in vivo comprising contacting a cell, such as a mammalian cell, with an effective amount of a compound of the invention.

The present invention further encompasses the incorporation of a compound of the invention into pharmaceutical compositions and single unit dosage forms useful in the treatment and prevention of a variety of diseases and disorders. Specific diseases and disorders include those responsive to the inhibition of antigen binding to an MHC class II HLA-DRB1*15:01 molecule, those responsive to the inhibition of antigen presentation by a MHC class II HLA-DRB1*15:01 molecule and those responsive to the inhibition of T cell proliferation. In a preferred embodiment, the MHC class II HLA-DR molecule is a MHC class II HLA-DRB1*15:01.

In one embodiment, the invention relates to a method for treating or preventing a disease responsive to the inhibition of antigen binding to a MHC class II HLA-DR molecule, particularly a MHC class II HLA-DRB1*15:01, comprising administering an effective amount of a compound of the invention to a patient in need thereof. In a preferred embodiment, the MHC class II HLA-DR molecule is MHC class II HLA-DRB1*15:01.

In another embodiment, the invention relates to a method for treating, preventing or preventing progression of a disease responsive to the inhibition of antigen presentation by a MHC class II HLA-DR molecule, particularly a MHC class II HLA-DRB1*15:01, comprising administering an effective amount of a compound of the invention to a patient in need thereof. In a preferred embodiment, the MHC class II HLA-DR molecule is MHC class II HLA-DRB1*15:01.

In another embodiment, the invention relates to a method for treating or preventing a disease responsive to the inhibition of T cell proliferation comprising administering an effective amount of a compound of the invention to a patient in need thereof. Particular diseases which the compounds of the invention are useful for treating or preventing include, but are not limited to: a genetic disease, a central nervous system (“CNS”) disease, an inflammatory disease, a neurodegenerative disease or an autoimmune disease.

In one embodiment, the disease is multiple sclerosis, which both a genetic disease due to its association with MHC class II, especially with HLA-DRB1*15:01 and is also an autoimmune disease with the HLA-DRB1*15:01 molecule recognizing an autoantigen.

In another embodiment, the genetic disease is anti-GBM (Goodpasture's disease).

In another embodiment, the genetic disease is lupus nephritis or PR3-ANCA.

Autoimmune diseases include those that affect only one organ or tissue type or may affect multiple organs and tissues. Organs and tissues commonly affected by autoimmune disorders include red blood cells, blood vessels, nerve cells and their insulation, connective tissues, endocrine glands (e.g., the thyroid or pancreas), muscles, joints, and skin. Examples of autoimmune diseases include, but are not limited to, encephalomyelitis, oophoritis, graft versus host disease, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue disease, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Ménière's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo and Wegener's granulomatosis. Thus, the invention encompasses the use of compounds of the invention to treat the diseases described above and herein.

In another embodiment, autoimmune diseases associated with antigen binding to MHC class II DRB1*15:01 are appropriate for treatment with compounds of the Formula I.

In one embodiment, the patient undergoes or has undergone a genetic screening process to determine the MHC class II allele that the patient has. In a particular embodiment, a patient has the MHC class II HLA-DRB1*15:01 allele.

In another embodiment, the patient has been diagnosed as having multiple sclerosis or symptoms of multiple sclerosis.

In another embodiment, the patient undergoes or has undergone a screening process to determine the presence of a cell in which normal cellular proteins are recognized as foreign, comprising the steps of screening a patient or a cell extracted therefrom by an acceptable T cell proliferation assay.

Specific methods of the invention further comprise the administration of an additional therapeutic agent (i.e., a therapeutic agent other than a compound of the invention). In certain embodiments of the present invention, a compound of the invention can be used in combination with at least one other therapeutic agent.

In particular, the invention provides combination therapies for prevention, treatment or amelioration of one or more symptoms associated with an autoimmune disease in a patient, said combination therapies comprising administering to said patient a compound of the invention, and at least one other prophylactic or therapeutic agent which has a different mechanism of action than the compound of the invention.

Therapeutic agents include, but are not limited to immunomodulatory agents, T cell and B cell receptor modulators, β-interferons, non-opioid analgesics, non-steroid anti-inflammatory agents, antiemetics, β-adrenergic blockers, anticonvulsants, antidepressants, Ca2+-channel blockers, anticancer agent or mixtures thereof.

Examples of immunomodulatory agents include, but are not limited to, methothrexate, leflunomide, cyclophosphamide, cyclosporine A, and macrolide antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), Copaxone® (glatiramer acetate).T cell receptor modulators, B-cell agents (ocrelizumab and the like) and cytokine receptor modulators.

Examples of T cell receptor modulators include, but are not limited to, anti-T cell receptor antibodies (e.g., anti-CD4 monoclonal antibodies, anti-CD3 monoclonal antibodies, anti-CD8 monoclonal antibodies, anti-CD40 ligand monoclonal antibodies, anti-CD2 monoclonal antibodies) and CTLA4-immunoglobulin.

Examples of β-interferons include, but are not limited to, Avonex® (interferon β-1a), Betaseron® (interferon (β-1b) and Rebif (interferon (β-1a).

Other therapeutic agents include fumarates (dimethyl fumarate, Tecfidera), VLA-4 inhibitors (Tysabri), sphingosine-1-phosphate-1 superagonists (Gilenya), and B-cell immunosuppressive agents (e.g., cladribine, Ocrevus)

The compounds of the invention and the other therapeutics agent can act additively or, more preferably, synergistically. In a preferred embodiment, a composition comprising a compound of the invention is administered concurrent with the administration of another therapeutic agent, which can be part of the same composition or in a different composition from that comprising the compounds of the invention. A preferred embodiment is the administration of a composition comprising a compound of the invention as a first-line therapy followed by administration of one of the other therapeutic agents when disease exacerbation is evident. The prophylactic or therapeutic agents of the combination therapies of the present invention can be administered concomitantly or sequentially to a patient. The prophylactic or therapeutic agents of the combination therapies of the present invention can also be cyclically administered. Cycling therapy involves the administration of a first prophylactic or therapeutic agent for a period of time, followed by the administration of a second prophylactic or therapeutic agent for a period of time and repeating this sequential administration, i.e., the cycle, in order to reduce the development of resistance to one of the agents, to avoid or reduce the side effects of one of the agents, and/or to improve the efficacy of the treatment.

The magnitude of a prophylactic or therapeutic dose of a particular active ingredient of the invention in the acute or chronic management of a disease or condition will vary, however, with the nature and severity of the disease or condition, and the route by which the active ingredient is administered. The dose, and perhaps the dose frequency, will also vary according to the age, body weight, and response of the individual patient. Suitable dosing regimens can be readily selected by those skilled in the art with due consideration of such factors.

In general, the recommended daily dose range for the conditions described herein lie within the range of from about 0.1 mg to about 3000 mg per day, given as a single once-a-day dose or as divided doses throughout a day. More specifically, the daily dose is administered in a single dose or in equally divided doses. Specifically, a daily dose range should be from about 1 mg to about 2500 mg per day, more specifically between about 10 mg and about 2000 mg per day, more specifically between about 50 mg and about 1500 mg per day, or as necessary to achieve effective concentrations at the site of action sufficient to block and maintain blockade of antigen presentation. This dose depends on the route of administration, bioavailability, metabolic stability, protein binding, and other factors known in the art. Compounds may also be administered in long-acting depot formulations that release effective amounts of the active ingredient over periods of several days to several weeks or months, usually following intramuscular or subcutaneous administration. In managing the patient, the therapy should be initiated at a lower dose, at about 1 mg per day to about 25 mg per day and increased if necessary up to about 200 mg per day to about 1000 mg per day, or to about 1500 mg per day to about 3000 mg per day, as either a single dose or divided doses, depending on the patient's global response.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

### Pharmaceutical Compositions

Pharmaceutical compositions and single unit dosage forms comprising a compound of the invention, or a pharmaceutically acceptable prodrug, salt, solvate or hydrate thereof, are also encompassed by the invention and methods of use disclosed herein. Individual dosage forms of the invention may be suitable for oral, mucosal (including sublingual, buccal, rectal, nasal, or vaginal), parenteral (including subcutaneous, intramuscular, bolus injection, intraarterial, or intravenous), transdermal, or topical administration.

Pharmaceutical compositions and dosage forms of the invention comprise a compound of the invention, or a pharmaceutically acceptable prodrug, salt, solvate or hydrate thereof. Pharmaceutical compositions and dosage forms of the invention typically also comprise one or more pharmaceutically acceptable excipients.

A particular pharmaceutical composition encompassed by this embodiment comprises a compound of the invention, or a pharmaceutically acceptable prodrug, salt, solvate or hydrate thereof, and at least one additional therapeutic agent. Examples of additional therapeutic agents include, but are not limited to immune suppressor agents, anti-cancer drugs and anti-inflammation therapies.

Single unit dosage forms of the invention are suitable for oral, mucosal (e.g., nasal, inhalation, sublingual, vaginal, buccal, or rectal), parenteral (e.g., subcutaneous, intravenous, bolus injection, infusion, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of inflammation or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more carriers, excipients or diluents. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In one embodiment, the compound of the invention is administered in a pharmaceutical formulation comprising carbonate.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise a compound of the invention, or a pharmaceutically acceptable prodrug, salt, solvate or hydrate thereof lie within the range of from about 0.1 mg to about 3000 mg per day, given as a single once-a-day dose in the morning but preferably as divided doses throughout the day taken with food. More specifically, the daily dose is administered twice daily in equally divided doses. Specifically, a daily dose range should be from about 1 mg to about 2500 mg per day, more specifically, between about 10 mg and about 2000 mg per day, more specifically, between about 25 mg and about 1500 mg per day, more specifically, between about 50 mg and about 1000 mg per day, more specifically, between about 100 mg and about 750 mg per day, more specifically, between about 200 mg and about 500 mg per day, more specifically, between about 250 mg and about 300 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response.

In one embodiment, the compounds of the invention are administered in a pharmaceutical composition comprising liposomes. The liposomes may be polymerized or unpolymerized and the compound of the invention may optionally be intercalated within the liposomes. Suitable lipophilic solvents or vehicles include fatty oils such as sesame oil, or synthetic fatty acid esters, such as ethyl oleate or triglycerides.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein, such as organic solvents including propylene glycol, polyethylene glycol, ethanol, glycerol, polyethylene glycol ricinoleate (Cremophor) or polyoxyethylene sorbitan fatty acid esters (Tween), can also be incorporated into the parenteral dosage forms of the invention. Parenteral solutions of the compounds of the invention can also comprise human serum proteins which serve as crystallization inhibitors, such as those described in U.S. Pat. No. 4,842,856, incorporated by referene herein in its entirety. Parenteral solutions of the compounds of the invention can further comprise poloxamers or polysorbates.

Parenteral dosage forms can also be administered in depot, long acting or slow-release forms comprising a compound of the invention in a matrix of a polymer of polyols and hydroxy carboxylic acids such as those disclosed in International Publication WO 78/00011, incorporated herein by reference in its entirety. Depot forms can also comprise a polyol ester containing polymeric-dicarboxylic acid residues (e.g. tartaric acid) such as those described in U.S. Pat. Nos.: 5,922,682 and 5,922,338, each of which is incorporated herein by reference in its entirety. Additional depot forms include matrices comprised of an ester of polyvinyl alcohol (M.W. of about 14000), polyethylene glycol (M.W. of about 6000 to 20,000) or polymer hydroxycarboxylic ester residues (e.g., lactic acid M.W. of about 26,000 to 114,000) or glycolic acid (M.W. of about 10,000), such as those disclosed in European application No. 92918, incorporated herein by reference in its entirety. Delayed release formulations for parenteral dosase forms also include binder-free granules as disclosed in U.S. Pat. No. 4,902,516 and those disclosed for use with vitamin D in U.S. Pat. No. 5,795,882, each incorporated by reference herein in its entirety.

Further parenteral dosage forms include wax microspheres such as those disclosed in U.S. Pat. No. 6,340,671, lipophilic formulations such as those disclosed in U.S. Pat. No. 6,335,346, non-acqueous compositions such as those disclosed in U.S. Pat. No. 5,965,603, carbohydrate polymers such as those disclosed in U.S. Pat. No. 5,456,922 and emulsions such as those disclosed in U.S. Pat. Nos. 4,563,354 and 5,244,925, each incorporated by reference herein in its entirety.

Parenteral dosages can be delivered via implantable devices, osmotic pumps, or catheter systems which are capable of delivering the composition at selectable rates (See U.S. Pat. Nos. 6,471,688; 6,436,091; 6,413,239; 6,464,688; 5,672,167; and 4,968,507, each incorporated by reference herein in its entirety).

### Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990) or Remington: The Science and Practice of Pharmacy, 20th ed., Lippincott, Williams and Wilkins, (2000).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Oral dosage forms containing excipients developed to enhance paracellular transport and to reduce enzymatic degradation in the gastrointestinal tract are also useful to increase oral bioavailability of compounds of the invention. One such technology is described by Maher S, et al. (2016) Drug Deliv Rev. 106(Pt B):277-319. Another such technology is described by Mehta N. et al (Mehta, Nozer & Stern, William & Carl, Stephen & Vrettos, John & Sturmer, Amy. (2013) Biopolymers, 23^{rd} American Peptide Symposium, 100: 237. Oral Delivery with PEPTELLIGENCE™: Examples of Preclinical and Clinical Studies with Peptides. 237-237.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof If used at all, lubricants are typically used in an amount of less than about 1 weight percent of the pharmaceutical compositions or dosage forms into which they are incorporated.

### Delayed Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, carboxymethyl cellulose, or other polymer matrices, gels, permeable membranes, osmotic systems, multilayer coatings, microparticles, liposomes, microspheres, or a combination thereof to provide the desired release profile in varying proportions. In a preferred embodiment, the controlled-release formulation is biodegradable.

Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled release. The compound of the invention may also be administered in a depot formulation or inclusion complex and can optionally be inserted under the skin.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

### Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 18th eds., Mack Publishing, Easton Pa. (1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to, acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### EXAMPLES

### Example 1

### Hydrolysis of Peptide Amide PV-267 to Peptide Acid PV-623

Following the general protocols outlined above, PV-267, having the sequence Ac-Val-Chg-Arg-Tic-Phe-NH2, was prepared as the acetate salt. 500 mg of PV-267 was stirred with 20 mL of 6 N HCl at room temperature overnight (some undissolved material remained). To the mixture acetonitrile (2 mL) was added to give a clear solution and the temperature was raised to 50° C. and kept at that temperature for 3 hr, resulting in complete conversion to the acid. The sample was lyophilized to give 550 mg of PV-623 (Ac-Val- Chg-Arg-Tic-Phe-OH) as the HCl salt. MS: M+H 762.

### Example 2

### Coupling of Peptide Acid (PV-623) to Alkoxyalkyl Substituted Amide (PV-3212)

A mixture of 160mg of PV-623 (0.16 mmol), 65 mg of HOAt (1-hydroxy-7-azabenzotriazole) (0.48 mmol), 127 μL of TMP (2,4,6-trimethylpyridine) (0.96 mmol), 71 μL of DIC (N,N′-diisopropylcarbodiimide) (0.48 mmol) and 42 μL of 2-methoxyethylamine (0.48 mmol) in 5 mL of dichloromethane was allowed to react at room temperature overnight. The mixture was evaporated and purified by prep HPLC to give 97 mg of PV-3212 (Ac-Val-Chg-Arg-Tic-Phe-OCH₂CH₂OCH₃) as the TFA salt (single peak on HPLC). MS: M+H 819,

### Example 3

### Coupling of Peptide Acid (PV-623) to Other Oligoalkoxyalkyl Substituted Amides

The above procedure was repeated with the oligoalkoxyalkyl amines shown in Table 8, using the same protocol (HOAt; TMP, DIC) to give the compounds shown in Table 5, above. The process may be used starting with other peptide amides to prepare other analogs based on the active sequences shown in Table 6 above.

TABLE 8

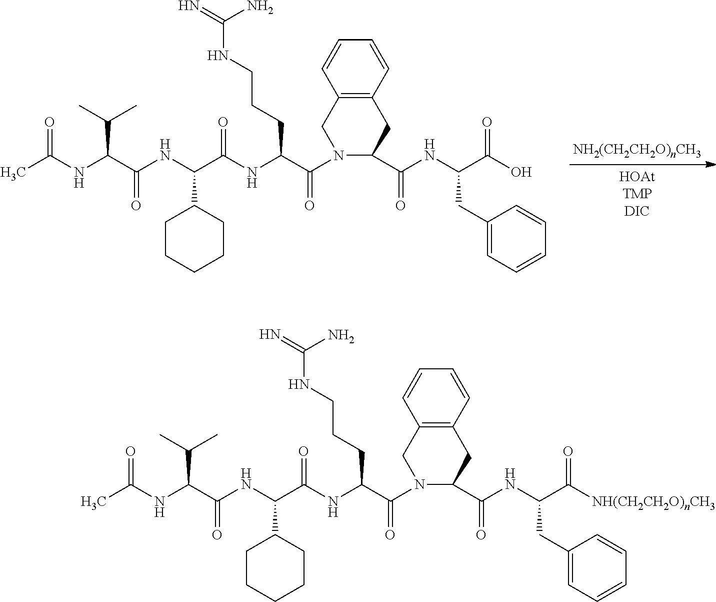

| PV | | |
|---|---|---|
| Num- | Oligoalkoxy | |
| ber | amine | Product |
| PV- 03212 | NH₂CH₂CH₂OCH₃ | 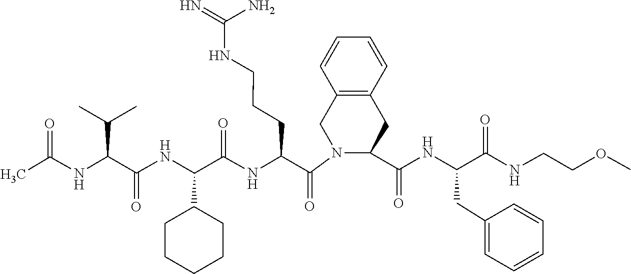 [SMILES: COCCNC(=O)[C@H](Cc1ccccc1)NC(=O)[C@@H]1Cc2ccccc2CN1C(=O)[C@H](CCCNC(=N)N)NC(=O)[C@@H](NC(=O)[C@@H](NC(C)=O)C(C)C)C1CCCCC1] |
| | | Ac-V-(Chg)-R-(Tic)-F—NH(CH₂CH₂O)₁CH₃ |
| PV- 03451 | NH₂(CH₂CH₂O)₂CH₃ | 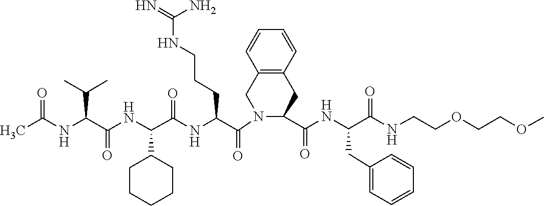 [SMILES: COCCOCCNC(=O)[C@H](Cc1ccccc1)NC(=O)[C@@H]1Cc2ccccc2CN1C(=O)[C@H](CCCNC(=N)N)NC(=O)[C@@H](NC(=O)[C@@H](NC(C)=O)C(C)C)C1CCCCC1] |
| | | Ac-V-(Chg)-R-(Tic)-F—NH(CH₂CH₂O)₂CH₃ |
| PV- 03449 | NH₂(CH₂CH₂O)₃CH₃ | 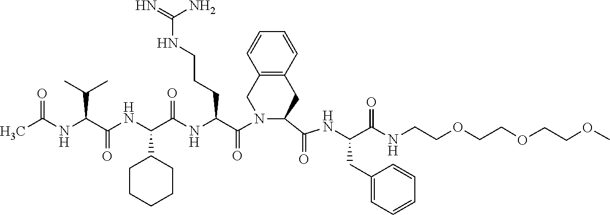 [SMILES: COCCOCCOCCNC(=O)[C@H](Cc1ccccc1)NC(=O)[C@@H]1Cc2ccccc2CN1C(=O)[C@H](CCCNC(=N)N)NC(=O)[C@@H](NC(=O)[C@@H](NC(C)=O)C(C)C)C1CCCCC1] |
| | | Ac-V-(Chg)-R-(Tic)-F—NH(CH₂CH₂O)₃CH₃ |
| PV- 03213 | NH₂(CH₂CH₂O)₄CH₃ | 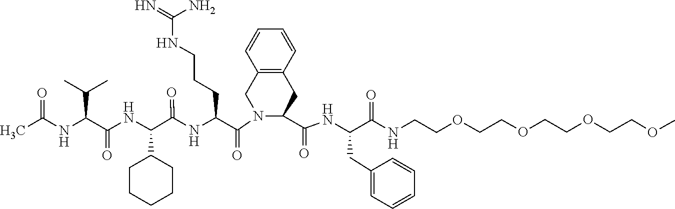 [SMILES: COCCOCCOCCOCCNC(=O)[C@H](Cc1ccccc1)NC(=O)[C@@H]1Cc2ccccc2CN1C(=O)[C@H](CCCNC(=N)N)NC(=O)[C@@H](NC(=O)[C@@H](NC(C)=O)C(C)C)C1CCCCC1] |
| | | Ac-V-(Chg)-R-(Tic)-F—NH(CH₂CH₂O)₄CH₃ |

The compounds in Table 8 were prepared by coupling the acid PV-623 with the appropriate oligoalkylamine using HOAt (1-hydroxy-7-azabenzotriazole), TMP (2,4,6-trimethylpyridine), and DIC (N,N′-diisopropylcarbodiimide) in dichloromethane at room temperature overnight. The mixture was evaporated and purified by prep HPLC eluting with 0.075% TFA in acetonitrile-water to give the compounds shown in Table 5.as their TFA salts.

### Example 4

### Preparation of Other Analogs (Table 6)

### General Method

Peptide sequences such as the sequences represented by the combinations in Table 6 and the specific analogs shown in Table 2, above, were produced by standard solid phase peptide synthesis to give the peptide amides. When the N-capping group is acetyl, they are converted by acid hydrolysis with 6N HCl to the corresponding acids which are, in turn, coupled with an oligoalkoxyalkyl amine. When the N-capping group is other than acetyl, the peptide is prepared on solid phase using a 2-chlorotrityl resin and N-capped with an acylating reagent (Boc protected in the case of the analogs with aminoacyl groups) and then cleaved from the resin without removing the Boc or other side chain protecting groups (e.g., Pbf on Arg) by treatment with HOAc/TFE/DCM(1:1:8) to get the fully-protected peptide-COOH (with Pbf on R). The peptide acid is then C-capped as above with the desired oligoalkoxyalkyl amine and this is fully deprotected with 95% TFA to give the desired compounds.

### Example 5

### Synthesis of NH₂-Gly-Val-Chg-Arg-Tic-Phe-NH(CH₂CH₂O)CH₃

Phenylalanine 2-chlorotrityl resin (ChemImpex) (100 mg) is sequentially coupled with FMOC-Tic, Pbf-Arg, Chg, and Val, then acylated with Boc-NH₂CH₂CO₂H (Boc-Gly). The fully protected peptide on resin is then cleaved from the resin with HOAc/TFE/DCM(1:1:8) to afford the fully-protected peptide-COOH. The protected peptide acid is then coupled with 2-methoxyethylamine as in Example 2, above, to give the C-capped, protected peptide. Deprotection of the N-Boc and Pbf groups with 95% TFA gives NH₂-Gly-Val-Chg-Arg-Tic-Phe-NH(CH₂CH₂O)CH₃.

### Example 6

### HLA II Binding Assay

EBV homozygous cell lines are used as sources of human HLA class II molecules. HLA-DR molecules are purified by affinity chromatography using the monomorphic mAb L243 (American Type Culture Collection, Manassas, Va.) coupled to protein A-Sepharose CL 4B gel (Amersham Pharmacia Biotech). The supernatant from lysed cells after centrifugation (100,000 g for 1 h) is applied to Sepharose 4B and protein A-Sepharose 4B columns and then to the specific antibody column. HLA DR molecules are eluted with 1.1 mM n-dodecyl β-D-maltoside, 500 mM NaCl and 500 mM Na₂CO₃ (pH 11.5). Fractions are immediately neutralized to pH 7 with 2 mM Tris-HCl (pH 6.8) and extensively dialyzed against 1 mM n-dodecyl β-D-maltoside, 150 mM NaCl, 10 mM phosphate (pH 7) buffer.

HLA-DR molecules are diluted in 10 mM phosphate, 150 mM NaCl, 1 mM n-dodecyl □-D-maltoside, 10 mM citrate, 0.003% thimerosal buffer with a biotinylated reference compound (biotinyl 6-aminocaproic-PKYVKQNTLKLAT for DRB1*0401 and DRB 1*0101 MHC class II molecules, botinyl 6-aminocaproic-EAEQLRRAYLDGTGVE for DRB 1*1501 and serial dilutions of competitor peptides and/or compounds of the invention. Samples (100 μl per well) are incubated in 96-wells polypropylene plates at 37° C. for 24 h to 72 h. After neutralization with 50 μl of 450 mM Tris HCl pH 7.5, 0.003% thimerosal, 0.3% BSA, 1 mM n-dodecyl β-D-maltoside buffer, samples are applied to 96-well maxisorp ELISA plates previously coated with 10 mg/ml L243 Mab and saturated with 100 mM Tris HCl pH=7.5, 0.3% BSA, and 0.003% thimerosal buffer. Samples are allowed to bind to the antibody-coated plates for 2 h at room temperature. Bound biotinylated compound is detected by incubating streptavidin-alkaline phosphatase conjugate, and after washings, by the addition of 4-methylumbelliferyl phosphate substrate. Emitted fluorescence is measured at 450 nm upon excitation at 365 nm on a Fluorolite 1000 fluorimeter. Maximal binding is determined by incubating the biotinylated peptide with the MHC class II molecule in the absence of competitor. Binding specificity is assessed by adding an excess of non-biotinylated peptide. Background does not significantly differ from that obtained by incubating the biotinylated peptide without MHC II molecules.

Other HLA alleles were assayed in a similar manner (Texier, et al., (2000), Journal of Immunology 164: 3177-3184).

HLA II molecules are purified on an affinity column from lysates of EBV cells that are homozygous for HLA genes. They are incubated in solution with a pre-determined concentration of a biotinylated peptide and different concentrations of a competitor test peptide. After 24 to 72 hours, the HLA II-peptide complexes are captured by anti-HLAII antibodies that were previously adsorbed onto an ELISA 96-well plate. The biotinylated peptide marker is detected by adding a streptavidine-enzyme conjugate. The degradation of the enzyme substrate produces a detectable fluorescent product. The binding properties of each peptide is characterized by its IC50, that is the concentration at which it inhibits 50% of marker peptide binding. Data for reference peptide binding to different HLA alleles is shown in Table 8

TABLE 8
HLA binding assay reference peptides and affinity

| HLA | Reference | Sequence | IC50, nM |
|---|---|---|---|
| DRB1*0101 | HA 306-318 | PKYVKQNTLKLAT | 5 |
| DRB1*0401 | HA 306-318 | PKYVKQNTLKLAT | 44 |
| DRB1*1101 | HA 06-318 | PKYVKQNTLKLAT | 38 |
| DRB1*0701 | YKL | AAYAAAKAAALAA | 34 |
| DRB1*0301 | MT T2-16 | AKTIAYDEEARRGLE | 100 |
| DRB1*1301 | B1 21-36 | TERVRLVTRHIYNREE | 330 |
| DRB1*1501 | A3 152-166 | EAEQLRRAYLDGTGVE | 14 |
| DRB5*0101 | HA 306-318 | PKYVKQNTLKLAT | 7 |
| DRB3*0101 | Lol 191-120 | ESWGAVWRIDTPDKLTGPFT | 5 |
| DRB4*0101 | E2/E168 | AGDLLAIETDKATI | 2 |
| DPB1*0401 | Oxy 271-287 | EKKYFAATQFEPLAARL | 10 |
| DPB1*0402 | Oxy 271-287 | EKKYFAATQFEPLAARL | 10 |

Data are expressed as the peptide concentration that prevents binding of 50% of the labeled peptide (IC₅₀).

Several compounds of the invention were assayed using the above protocol to demonstrate their selectivity for particular DR molecules as set forth in Table 2 above.

### Example 7

### T-Cell Proliferation Inhibition Assay

The compounds of the present invention can be tested for inhibition of T-cell response. One skilled in the art would be familiar with many of the known techniques used to measure T-cell proliferation. See Bolin, D., (2000) J. Med. Chem. 43:2135-2148; Chirathaworn, C. (2002) J. Immunol. 168(11):5530-5537; Falcioni, F., et. al. (1999) Nature Biotechnology 17:562-567.

Mitomycin C—treated (150 g/ml, 37° C., 60 min.) APCs are preincubated with a stimulatory concentration of a compound of the invention in 96-well U bottom plates (4×10⁴ LBL or 10⁵ DR-transgenic spleen cells/well) at 37° C. for 2 h. T cells (2×10⁴/well) are then added and the cells are cultured for 3 days. Proliferative T-cell response is measured by [³ H]thymidine incorporation, using a liquid scintillation counter.

For compound screening, HEL-specific polyconal T-cell lines and OVA-specific T-cell hybridomas are derived from HLA-DR4-IE chimeric, transgenic mice, and splenocytes of the same transgenic mice serve as APCs. Bolin, D. Id. T-cell inhibitory potency can be measured relative to the IC₅₀ of a model peptide or of a compound with known inhibitory activity. Alternatively, standard control compounds identified in the art can be used, e.g., phorbol (10 nM) in combination with ionomycin (0.5 □M).

T-cell activation may also be measured using release of interferon-gamma from

### Example 8

### Cathepsin Stability Assay

The reference peptide (Ac(Cha)RAMASL-NH₂) is incubated with a buffered solution comprising cathepsin B, D, or L at about 37° C. at pH 6. Degradation products are resolved using reverse phase HPLC (acetonitrile/water/TFA gradient elution). The height of the parent peak is followed as a function of incubation time with the enzyme and plotted relative to an internal standard peak height. The mass of selected peaks is determined to identify cleavage sites. The same procedure is followed to assess the stability of the compounds of Formula I or the specific compounds in Table 2.

### Example 9

### Human Cytokine ELISPOT Assays

Primary anti-human IFN-γ mAb (Thermo Scientific; 2G1) was diluted in PBS and coated on ELISPOT plate (Millipore) at 4° C., overnight. After washing and blocking, autologous APCs (irradiated PBMCs) pretreated with different concentrations of the DRB1*15:01 inhibitors for 45 min were added on the plate (0.3−0.5×10⁶ cells per well), followed by the addition of Ag or positive control using PHA (Sigma-Aldrich) or anti-human CD3 mAb (Bio X Cell; OKT3) along with BC3 T cells or enriched PBMC T cells (4,000-20,000 cells per well). Plates were incubated at 37° C. for 24 h, then washed and biotinylated anti-human IFN-γ mAb (Thermo Scientific; B133.5) was added at 4° C., overnight. After that, plates were incubated with Streptavidin AP (Invitrogen) and developed with BCIP/NBT Phosphatase substrate (KPL) after washing. After developing of the plates, image analysis of ELISPOT assays was performed on a Series 2 ImmunoSpot analyzer and software (CTL) as described previously (Kawamura, K., et al., (2008) J. Immunol. 181: 3202-3211).

### ADDITIONAL REFERENCES

Ji N, Somanaboeina A, Dixit A, Kawamura K, Hayward N J, Self C, Olson G L, Forsthuber T G. “Small molecule inhibitor of antigen binding and presentation by HLA-DR2b as a therapeutic strategy for the treatment of multiple sclerosis.” J Immunol. 2013 Nov 15;191(10):5074-84. (cited by others)

Olson, et al., “Inhibition of Antigen Presentation by MHC Class II Molecules and Methods of Use Thereof,” U.S. Pat. No. 7,439,231.

Olson, et. al., “Methods of Use of Inhibitors of Inhibition of Antigen Presentation by MHC Class II Molecules,” U.S. Pat. No. 8,222,215.

Olson, et al., “Inhibitors of Antigen Presentation by MHC Class II Molecules,” U.S. Pat. No. 8,598,312.

Huynh M., et al., “HLA-DR15-specific inhibition attenuates autoreactivity to the Goodpasture antigen.” J Autoimmun. 2019 103:102276.

Falcioni et al., “Peptidomimetic compounds that inhibit antigen presentation by autoimmune disease-associated class II major histocompatibility molecules,” Nature Biotechnology 17:562-567 (1999) (cited by others)

Lehmann et al., “Selective peptidomimetic blockers of autoantigen presentation: a novel therapeutic approach to autoimmune disease,” Trends in Pharmacological Sciences (Elsevier, Haywarth, G B) 21(3):79-80. (cited by others)
Adorini, et al., (1988), “In Vivo Competition Between Self Peptides and Foreign Antigens in T-cell Activation”, Nature, 334, 623-625. (cited by others)
Bolin, et al., (2000), “Peptide and Peptide Mimetic Inhibitors of Antigen Presentation by HLA-DR Class II MEW Molecules, Design, Structure-Activity Relationships, and X-ray Crystal Structures”, J. Med. Chem., 43, 2135-2148. (cited by others)
Giordano, M. et al. (2002), “Genetics of Multiple Sclerosis—Linkage and Association Studies”, Am. J. Pharmacogenomics 2:37-58. (cited by others)
Hammer, J. et al. (1993), “Promiscuous and Allele-Specific Anchors in HLA-DR-Binding Peptides”, Cell 74:197-203. (cited by others)
Ishioka, et al., (1994), “Failure to Demonstrate Long-Lived MHC Saturation Both In Vitro and In Vivo”, J. Immunol. 152, 4310-4319. (cited by others)
Lamont, et al. (1990), “The Use of Peptide Analogs with Improved Stability and MHC Binding Capacity to Inhibit Antigen Presentation In Vitro and In Vivo”, J. Immunol. 144, 2493-2498. (cited by others)
Smith et al. (1998), “Crystal Structure of HLA-DR2 (DRA*0101,DRB1*1501) Complexed with a Peptide from Human Myelin Basic Protein”, J. Exp. Med. 188:1511-1520. (cited by others)
Stern et al. (1994), “Crystal Structure of the Human Class II MHC Protein HLA-DR1 Complexed with an Influenza Virus Peptide”, Nature 368:215-221. (cited by others)
Wucherpfennig et al. (1994), “Structural Requirements for Binding of an Immunodominant Myelin Basic Protein Peptide to DR2 Isotypes and for Its Recognition by Human T Cell Clones”, J. Exp. Med. 179:279-290. (cited by others)

While the invention has been described with respect to the particular non-limiting embodiments, it will be apparent to those skilled in the art that various changes and modifications can be made without departing from the spirit and scope of the invention as defined in the claims. Such modifications are also intended to fall within the scope of the appended claims.

1. A compound of the formula (I):
K—P₁—P₂—P₃—P₄—P₅—K₂ (I),
or a pharmaceutically acceptable salt thereof,
wherein:
K₁ is an N-capping group;
P₁-P₅ are substituents that, when present, are coupled by amide bonds and are each independently amino acyl groups of a natural amino acid or a non-natural amino acid; and
K₂ is a substituent in which the C-terminus is an oligoalkoxyalkyl amide group.

2. The compound according to claim 1, wherein K₂ is NH-(alkoxy)ₙ-alkyl wherein n is 1-4.

3. The compound according to claim 2 wherein K₂ is NHCH₂CH₂OCH₃, NH(CH₂CH₂O)₂CH₃, NH(CH₂CH₂O)₃CH₃, or NH(CH₂CH₂O)₄CH₃.

4. The compound according to claim 1 wherein K₁ is acyl or aminoacyl and K₂ is NH-(alkoxy)ₙ-alkyl, wherein n is 1-4.

5. The compound according to claim 3, wherein K₁ is acetyl.

6. The compound according to claim 1, comprising the combinations of Table 6.

7. The compound according to claim 1, wherein:
K₁ is acetyl;
P₁-P₂-P₃-P₄-P₅ is Val-Chg-Arg-Tic-Phe; and
K₂ is NH—(CH₂CH₂O)ₙCH₃wherein n is 0-10.

8. The compound according to claim 2 of the formula Ac-V-(Chg)-R-(Tic)-F-NHCH₂ CH₂OCH₃.

9. The compound according to claim 2 of the formula Ac-V-(Chg)-R-(Tic)-F-NH(CH₂CH₂O)₂CH₃.

10. Th compound according to claim 2 of the formula Ac-V-(Chg)-R-(Tic)-F-NH(CH₂CH₂O)₃CH₃.

11. The compound according to claim 2 of the formula Ac-V-(Chg)-R-(Tic)-F-NH(CH₂CH₂O)₄CH₃.

12. A compound of formula (II):
K₁-(B)-Chg-R-Tic-(ArAA)-K₂ (II),
or a pharmaceutically acceptable salt thereof,
wherein:
(B) is an alpha branched amino acid;
K₁ is an acyl or aminoacyl group;
ArAA is an aralkyl-L-amino acid or a 2-Amino-3-phenylpropanoic acid; and
K₂ is NH-(oligoalkoxy)alkyl or NH-(alkyl-O)ₙ-alkyl, wherein n=1-10.

13. The compound according to claim 12, wherein (B) is selected from the group consisting of t-BuG (alpha-tert-butylglycine; tert-Leucine), Val, Ile, Nva and Ala.

14. The compound according to claim 12, wherein K₁ is selected from the group consisting of CH₃CO, NH₂(CH₂)CO, NH₂(CH₂)₂CO, NH₂(CH₂)₃CO and CH₃NHCH₂CO.

15. The compound according to claim 12, wherein ArAA is selected from the group consisting of Phe, 4-F-Phe, Trp, 4-Indolyl-Ala and 3-Benzothienyl-Ala.

16. The compound according to claim 12, wherein K₂ is selected from the group consisting of NHCH₂CH₂OCH₃, NH(CH₂CH₂O)₂CH₃, NH(CH₂CH₂O)₃CH₃ and NH(CH₂CH₂O)₄CH₃.

17. The compound according to claim 1, wherein K₁ is selected from the group consisting of alkyl-C(O)-, hydroxyalkyl-C(O)-, aralkyl-C(O)-, heteroarylalkyl-C(O), alkoxy-C(O)-, alkoxycarbonylalkyl-C(O)-, amino-C(O)-, monoalkylamino-C(O)-, dialkylamino-C(O)-, aminoalkyl-C(O)-, monoalkylaminoalkyl-C(O)-, dialkylaminoalkyl-C(O)-, NH₂(CH₂)₄C(O)-, NH₂(CH₂)₃C(O)-, hydroxyalkyl, sulfonyl, aminosulfonyl, alkylaminosulfonyl, dialkylaminosulfonyl, or arylsulfonyl.

18. The compound according to claim 1, wherein P₁-P₅ are each independently amino acyl groups of a natural amino acid or a non-natural amino acid including those set forth in Table 6, above, wherein at least one of P₁-P₅, or at least two of P₁-P₅, or at least three of P₁-P₅ is a non-natural amino acid or peptide mimetic.

19. The compound according to claim 1, wherein K₂ is NH-(alkoxy)ₙ-alkyl wherein n is 1-10.

20. A pharmaceutical composition, comprising a therapeutically effective amount of a compound according to claim 1 or claim 12, or a pharmaceutically acceptable salt thereof, and a pharmaceutically acceptable carrier.

21. A method for the treatment of an autoimmune disease or disorder associated with antigen presentation by MHC Class II HLA-DRB1*15:01, comprising the step of administering a therapeutically effective amount of a compound according to claim 1 or claim 12, or a pharmaceutically acceptable salt thereof, to a patient in need thereof.
